(19) United States
(12) Patent Application Publication
(10) Pub. No.: US 2010/0137312 A1
(43) Pub. Date: Jun. 3, 2010

(54) NOVEL AROMATIC HETEROCYCLIC CARBOXYLIC ACID AMIDE DERIVATIVES USEFUL AS POTASSIUM CHANNEL MODULATORS

(75) Inventors: Antonio Nardi, Ballerup (DK); Jeppe Kejser Christensen, Kobenhavn (DK); Palle Christophersen, Ballerup (DK); David Spencer Jones, Smorum (DK); Elsebet Østergaard Nielsen, kobenhavn (DK); Dorte Strøbæk, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: neuroSearch A/S, Ballerup (DK)

(21) Appl. No.: 12/600,159

(22) PCT Filed: May 13, 2008
(86) PCT No.: PCT/EP08/55803

§ 371 Date: Jan. 15, 2010

Related U.S. Application Data
(60) Provisional application No. 60/938,032, filed on May 15, 2007.

(30) Foreign Application Priority Data
May 15, 2007 (DK) PA 2007 00731

(51) Int. Cl. A61K 31/41; C07D 405/02; C07D 403/02; C07D 307/02; C07D 413/02; C07D 413/14; A61K 31/34; A61K 31/4245; A61K 31/454; A61K 31/5377; A61P 25/00; A61P 35/00; A61P 1/00; A61P 29/00

(52) U.S. Cl. 514/236.2; 548/253; 549/484; 548/131; 546/209; 544/138; 514/382; 514/381; 514/471; 514/364; 514/326

(57) ABSTRACT

This invention relates to novel aromatic heterocyclic carboxylic acid amide derivatives of formula (I) that are found to be potent modulators of potassium channels and, as such, are valuable candidates for the treatment of diseases or disorders as diverse as those which are responsive to the modulation of potassium channels.

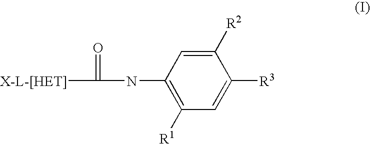

### TECHNICAL FIELD

This invention relates to novel aromatic heterocyclic carboxylic acid amide derivatives that are found to be potent modulators of ion channels and, as such, are valuable candidates for the treatment of diseases or disorders as diverse as those which are responsive to the modulation of ion channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

WO 2007/044724 describes certain N-tetrazolylphenyl carboxamide derivatives useful as PIM-1 and PIM-3 protein kinase inhibitors. However, the aromatic heterocyclic carboxylic acid amide derivatives of the present invention are not described, and their use as potassium channel modulators certainly not suggested.

### SUMMARY OF THE INVENTION

It is an object of the invention to provide novel aromatic heterocyclic carboxylic acid amide derivatives useful as ion channel modulators. The aromatic heterocyclic carboxylic acid amide derivatives of the invention may be characterised by Formula I

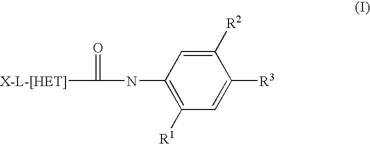

a stereoisomer or a mixture of its stereoisomers, or a pharmaceutically-acceptable addition salt thereof, wherein

X represents alkyl, cycloalkyl, alkenyl, alkynyl, methoxyiminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl, piperidinyl, morpholin-4-yl and pyridinyl, which phenyl and heterocyclic groups are optionally substituted one or more times with substituents selected from the group consisting of alkyl, halo, trifluoromethyl, cyano, hydroxy, phenyl and alkoxy-carbonyl;

L is absent (i.e. representing a covalent bond), or represents the linking group —CH₂— or —CONH—;

HET represents a five-membered aromatic heterocyclic group selected from furanyl, thienyl, oxazolyl, thiazolyl, pyrazolyl and triazolyl, which heterocyclic group is optionally substituted one or more times with substituents selected from the group consisting of alkyl, halo, trifluoromethyl and cyano;

R¹ represents tetrazolyl, tetrazolyl-alkoxy, N-phenyl-carbamoyl, alkyl-sulfonyl-amino-carbonyl, N-alkyl-sulfonyl-carboxamide, N-phenyl-sulfonyl-carboxamide, carboxy, N-cyano-carboxamide, sulfamoyl, sulfonic acid, sulfonic acid alkyl ester, sulfonic acid phenyl ester, or a oxadiazolyl oxo- or thio-derivative selected from 5-oxo-4,5-dihydro-[1,2,4]oxadiazol-3-yl and 5-thioxo-4,5-dihydro-[1,2,4]oxadiazol-3-yl;

R² represents halo or trifluoromethyl; and

R³ represents hydrogen, halo, trifluoromethyl, amino, N-alkyl-amino, N,N-dialkyl-amino, piperidinyl or morpholinyl.

In another aspect the invention provides pharmaceutical compositions comprising a therapeutically effective amount of an aromatic heterocyclic carboxylic acid amide derivative of the invention.

In a third aspect the invention relates to the use of the aromatic heterocyclic carboxylic acid amide derivatives of the invention for the manufacture of pharmaceutical compositions.

In a further aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the aromatic heterocyclic carboxylic acid amide derivative of the invention.

Other objects of the invention will be apparent to the person skilled in the art from the following detailed description and examples.

### DETAILED DISCLOSURE OF THE INVENTION

In its first aspect the invention provides novel aromatic heterocyclic carboxylic acid amide derivatives of Formula I

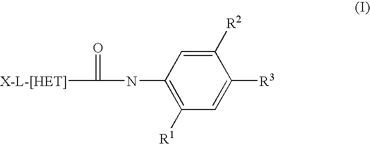

a stereoisomer or a mixture of its stereoisomers, or a pharmaceutically-acceptable addition salt thereof, wherein

X represents alkyl, cycloalkyl, alkenyl, alkynyl, methoxyiminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl, piperidinyl, morpholin-4-yl and pyridinyl, which phenyl and heterocyclic groups are optionally substituted one or more times with substituents selected from the group consisting of alkyl, halo, trifluoromethyl, cyano, hydroxy, phenyl and alkoxy-carbonyl;

L is absent (i.e. representing a covalent bond), or represents the linking group —CH₂— or —CONH—;

HET represents a five-membered aromatic heterocyclic group selected from furanyl, thienyl, oxazolyl, thiazolyl, pyrazolyl and triazolyl, which heterocyclic group is optionally substituted one or more times with substituents selected from the group consisting of alkyl, halo, trifluoromethyl and cyano;

R¹ represents tetrazolyl, tetrazolyl-alkoxy, N-phenyl-carbamoyl, alkyl-sulfonyl-amino-carbonyl, N-alkyl-sulfonyl-carboxamide, N-phenyl-sulfonyl-carboxamide, carboxy, N-cyano-carboxamide, sulfamoyl, sulfonic acid, sulfonic acid alkyl ester, sulfonic acid phenyl ester, or a oxadiazolyl oxo- or thio-derivative selected from 5-oxo-4,5-dihydro-[1,2,4]oxadiazol-3-yl and 5-thioxo-4,5-dihydro-[1,2,4]oxadiazol-3-yl;

R² represents halo or trifluoromethyl; and

R³ represents hydrogen, halo, trifluoromethyl, amino, N-alkyl-amino, N,N-dialkyl-amino, piperidinyl or morpholinyl.

In a preferred embodiment the aromatic heterocyclic carboxylic acid amide derivative of the invention is a compound of Formula I, or a pharmaceutically-acceptable addition salt thereof, wherein X represents alkyl, cycloalkyl, alkenyl, alkynyl, methoxy-iminomethyl (i.e. a substituent of formula CH₃O—N═C—), amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl, piperidinyl, morpholin-4-yl and pyridinyl, which phenyl and heterocyclic groups are optionally substituted one or more times with substituents selected from the group consisting of alkyl, halo, trifluoromethyl, cyano, hydroxy, phenyl and alkoxy-carbonyl.

In a more preferred embodiment X represents alkyl, alkenyl, alkynyl, methoxy-iminomethyl (i.e. a substituent of formula CH₃O—N═C—), amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl and morpholin-4-yl, which phenyl and heterocyclic groups are optionally substituted one or more times with substituents selected from the group consisting of alkyl, halo, trifluoromethyl, cyano, hydroxy and phenyl.

In another more preferred embodiment X represents alkyl, cycloalkyl, alkenyl, alkynyl, methoxyiminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino or aryl-carbonyl-amino.

In a third more preferred embodiment X represents alkyl, alkenyl, alkynyl, methoxyiminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino or aryl-carbonyl-amino.

In a fourth more preferred embodiment X represents alkyl or cycloalkyl.

In a fifth more preferred embodiment X represents cycloalkyl and in particular cyclopentyl.

In a sixth more preferred embodiment X represents phenyl or a five- or six-membered heterocyclic group selected from imidazolyl, 2-oxopyrrolidin-1-yl, piperidinyl, morpholin-4-yl and pyridinyl, which phenyl and heterocyclic groups are optionally substituted one or more times with substituents selected from the group consisting of alkyl, halo, trifluoromethyl, cyano, hydroxy, phenyl and alkoxy-carbonyl.

In a seventh more preferred embodiment X represents phenyl or a five- or six-membered heterocyclic group selected from imidazolyl, 2-oxopyrrolidin-1-yl and morpholin-4-yl, which phenyl and heterocyclic groups are optionally substituted one or more times with substituents selected from the group consisting of alkyl, halo, trifluoromethyl, cyano, hydroxy and phenyl.

In an eighth more preferred embodiment X represents phenyl, piperidinyl, morpholin-4-yl or pyridinyl, which phenyl and heterocyclic groups are optionally substituted with halo, trifluoromethyl or alkoxy-carbonyl.

In a ninth more preferred embodiment X represents phenyl, piperidinyl or pyridinyl, which phenyl, piperidinyl and pyridinyl groups are optionally substituted with halo, trifluoromethyl or alkoxy-carbonyl.

In a tenth more preferred embodiment X represents phenyl, piperidinyl, morpholin-4-yl or pyridinyl, which phenyl and heterocyclic groups are optionally substituted with chloro, bromo, trifluoromethyl or isobutoxy-carbonyl.

In an eleventh ninth more preferred embodiment X represents phenyl, piperidinyl, morpholin-4-yl or pyridinyl, which phenyl and heterocyclic groups are optionally substituted with chloro, bromo, trifluoromethyl or isobutoxy-carbonyl.

In a twelfth more preferred embodiment X represents phenyl, which phenyl is optionally substituted one or two times with substituents selected from the group consisting of alkyl, halo, trifluoromethyl, cyano, hydroxy and phenyl.

In a thirteenth more preferred embodiment X represents phenyl substituted with halo, trifluoromethyl, cyano, hydroxy or phenyl.

In a fourteenth more preferred embodiment X represents phenyl substituted with halo, in particular chloro or bromo, or trifluoromethyl.

In a fifteenth more preferred embodiment X represents a five- or six-membered heterocyclic group selected from imidazolyl, 2-oxopyrrolidin-1-yl and morpholin-4-yl, which heterocyclic group is optionally substituted with alkyl, halo, trifluoromethyl, cyano, hydroxy or phenyl.

In a sixteenth more preferred embodiment X represents morpholin-4-yl.

In a seventeenth more preferred embodiment X represents an imidazolyl group, which imidazolyl group is optionally substituted with alkyl, halo, trifluoromethyl, cyano, hydroxy or phenyl.

In an eighteenth more preferred embodiment X represents a 2-oxopyrrolidin-1-yl group, which 2-oxopyrrolidin-1-yl group is optionally substituted with alkyl, halo, trifluoromethyl, cyano, hydroxy or phenyl.

In a nineteenth more preferred embodiment X represents a morpholin-4-yl group, which morpholin-4-yl group is optionally substituted with alkyl, halo, trifluoromethyl, cyano, hydroxy or phenyl.

In another preferred embodiment the aromatic heterocyclic carboxylic acid amide derivative of the invention is a compound of Formula I, or a pharmaceutically-acceptable addition salt thereof, wherein L is absent (i.e. representing a covalent bond), or represents the linking group —CH₂— or —CONH—.

In a more preferred embodiment L is absent (i.e. representing a covalent bond), or represents the linking group —CH₂—.

In another more preferred embodiment L is absent (i.e. represents a covalent bond).

In a third more preferred embodiment L represents the linking group —CH₂—.

In a fourth more preferred embodiment L represents the linking group —CONH—.

In a third preferred embodiment the aromatic heterocyclic carboxylic acid amide derivative of the invention is a compound of Formula I, or a pharmaceutically-acceptable addition salt thereof, wherein HET represents a five-membered aromatic heterocyclic group selected from furanyl, thienyl, oxazolyl, thiazolyl, pyrazolyl and triazolyl, which heterocyclic group is optionally substituted one or more times with substituents selected from the group consisting of alkyl, halo and trifluoromethyl.

In a more preferred embodiment HET represents a five-membered aromatic heterocyclic group selected from furanyl, oxazolyl, thiazolyl, pyrazolyl and triazolyl, which heterocyclic group is optionally substituted one or more times with substituents selected from the group consisting of alkyl, halo and trifluoromethyl.

In another more preferred embodiment HET represents furanyl, optionally substituted one or more times with substituents selected from the group consisting of alkyl, in particular methyl, halo and trifluoromethyl.

In a third more preferred embodiment HET represents oxazolyl, optionally substituted one or more times with substituents selected from the group consisting of alkyl, in particular methyl, halo and trifluoromethyl.

In a fourth more preferred embodiment HET represents thiazolyl, optionally substituted one or more times with substituents selected from the group consisting of alkyl, in particular methyl, halo and trifluoromethyl.

In a fifth more preferred embodiment HET represents pyrazolyl optionally substituted one or more times with substituents selected from the group consisting of alkyl, in particular methyl, halo and trifluoromethyl.

In a sixth more preferred embodiment HET represents a triazolyl group, and in particular 1,2,3-triazolyl, which heterocyclic group is optionally substituted one or more times with substituents selected from the group consisting of alkyl, in particular methyl, halo and trifluoromethyl.

In a seventh more preferred embodiment HET represents a five-membered aromatic heterocyclic group selected from furan-2,4-diyl, thien-2,4-diyl, oxazol-2,4-diyl, thiazol-2,4-diyl, pyrazol-1,4-diyl and 1,2,3-triazol-1,4-diyl, which heterocyclic group is optionally substituted one or more times with substituents selected from the group consisting of alkyl, in particular methyl, halo and trifluoromethyl.

In an even more preferred embodiment the five-membered aromatic heterocyclic group is optionally substituted with alkyl, in particular methyl, halo or trifluoromethyl.

In a still more preferred embodiment the five-membered aromatic heterocyclic group is optionally substituted with alkyl, in particular methyl, or trifluoromethyl.

In a yet more preferred embodiment the five-membered aromatic heterocyclic group is optionally substituted with methyl or trifluoromethyl.

In a fourth preferred embodiment the aromatic heterocyclic carboxylic acid amide derivative of the invention is a compound of Formula I, or a pharmaceutically-acceptable addition salt thereof, wherein R¹ represents tetrazolyl, tetrazolyl-alkoxy, N-phenyl-carbamoyl, alkyl-sulfonyl-amino-carbonyl, N-alkyl-sulfonyl-carboxamide, N-phenyl-sulfonyl-carboxamide, carboxy, N-cyano-carboxamide, sulfamoyl, sulfonic acid, sulfonic acid alkyl ester, sulfonic acid phenyl ester, or a oxadiazolyl oxo- or thio-derivative selected from 5-oxo-4,5-dihydro-[1,2,4]oxadiazol-3-yl and 5-thioxo-4,5-dihydro-[1,2,4]oxadiazol-3-yl.

In a more preferred embodiment Fe represents a tetrazolyl group, and in particular 1H-tetrazol-5-yl.

In another more preferred embodiment R¹ represents a tetrazolyl-alkoxy group, and in particular 1H-tetrazol-5-yl-methoxy.

In a third more preferred embodiment R¹ represents N-phenyl-carbamoyl.

In a fourth more preferred embodiment R¹ represents an alkyl-sulfonyl-amino-carbonyl group, and in particular methyl-sulfonyl-amino-carbonyl.

In a fifth more preferred embodiment R¹ represents an N-alkyl-sulfonyl-carboxamide group, and in particular N-methyl-sulfonyl-carboxamide.

In a sixth more preferred embodiment R¹ represents N-phenyl-sulfonyl-carboxamide.

In a seventh more preferred embodiment R¹ represents carboxy.

In an eight more preferred embodiment R¹ represents N-cyano-carboxamide.

In a ninth more preferred embodiment R¹ represents sulfamoyl.

In a tenth more preferred embodiment R¹ represents sulfonic acid.

In an eleventh more preferred embodiment R¹ represents a sulfonic acid alkyl ester, and in particular sulfonic acid methyl ester.

In a twelfth more preferred embodiment R¹ represents sulfonic acid phenyl ester.

In a thirteenth more preferred embodiment R¹ represents 5-oxo-4,5-dihydro-[1,2,4]oxadiazol-3-yl.

In a fourteenth more preferred embodiment R¹ represents 5-thioxo-4,5-dihydro-[1,2,4]oxadiazol-3-yl.

In a fifth preferred embodiment the aromatic heterocyclic carboxylic acid amide derivative of the invention is a compound of Formula I, or a pharmaceutically-acceptable addition salt thereof, wherein R² represents halo or trifluoromethyl.

In a more preferred embodiment R² represents halo.

In an even more preferred embodiment R² represents fluoro or chloro.

In a sixth preferred embodiment the aromatic heterocyclic carboxylic acid amide derivative of the invention is a compound of Formula I, or a pharmaceutically-acceptable addition salt thereof, wherein R³ represents hydrogen, halo, trifluoromethyl, amino, N-alkyl-amino, N,N-dialkyl-amino, piperidinyl or morpholinyl.

In a more preferred embodiment R³ represents hydrogen, halo or trifluoromethyl.

In another more preferred embodiment R³ represents hydrogen, halo, N,N-dialkyl-amino, piperidinyl or morpholinyl.

In a more preferred embodiment R³ represents hydrogen or halo.

In an even more preferred embodiment R³ represents hydrogen.

In a still more preferred embodiment R³ represents halo, in particular fluoro.

In another more preferred embodiment R³ represents hydrogen or fluoro.

In a further more preferred embodiment R³ represents N,N-dialkyl-amino, and in particular N,N-dimethyl-amino; piperidinyl or morpholinyl.

In a most preferred embodiment the aromatic heterocyclic carboxylic acid amide derivative of the invention is
5-(4-Chloro-phenyl)-2-trifluoromethyl-furan-3-carboxylic acid [5-chloro-2-(1H-tetrazol-5-yl)-phenyl]-amide;
1-(4-Chloro-phenyl)-5-trifluoromethyl-1H-pyrazole-4-carboxylic acid [5-chloro-2-(1H-tetrazol-5-yl)-phenyl]-amide;
1-(4-Bromo-benzyl)-5-methyl-1H-[1,2,3]triazole-4-carboxylic acid [5-chloro-2-(1H-tetrazol-5-yl)-phenyl]-amide;
5-(4-Chloro-phenyl)-2-trifluoromethyl-furan-3-carboxylic acid (5-chloro-2-phenylcarbamoyl-phenyl)-amide;
5-(4-Chloro-phenyl)-2-trifluoromethyl-furan-3-carboxylic acid (5-chloro-2-methanesulfonylaminocarbonyl-phenyl)-amide;
4-Chloro-2-{[5-(4-chloro-phenyl)-2-trifluoromethyl-furan-3-carbonyl]-amino}-benzenesulfonic acid;
5-(4-Chloro-phenyl)-2-trifluoromethyl-furan-3-carboxylic acid [5-chloro-2-(1H-tetrazol-5-ylmethoxy)-phenyl]-amide;
4,5-Dichloro-2-{[5-(4-chloro-phenyl)-2-trifluoromethyl-furan-3-carbonyl]amino}-benzenesulfonic acid;
4-Methyl-2-(4-trifluoromethyl-phenyl)-thiazole-5-carboxylic acid [5-chloro-2-(1H-tetrazol-5-yl)-phenyl]-amide;
4-Chloro-2-{[5-(4-chloro-phenyl)-2-trifluoromethyl-furan-3-carbonyl]-amino}-benzoic acid;
4-Chloro-2-{[5-(4-chloro-phenyl)-2-trifluoromethyl-furan-3-carbonyl]-amino}-benzoylcyanamide;
5-(4-Chloro-phenyl)-2-methyl-furan-3-carboxylic acid [5-chloro-2-(1H-tetrazol-5-yl)-phenyl]-amide;
2-{[5-(4-Chloro-phenyl)-2-trifluoromethyl-furan-3-carbonyl]-amino}-4,5-difluoro-benzoic acid;
4,5-Dichloro-2-{[5-(4-chloro-phenyl)-2-trifluoromethyl-furan-3-carbonyl]-amino}-benzenesulfonic acid methyl ester;
5-Methyl-2-phenyl-2H-[1,2,3]triazole-4-carboxylic acid [5-chloro-2-(1H-tetrazol-5-yl)-phenyl]-amide;
5-Trifluoromethyl-2-(4-trifluoromethyl-phenyl)-oxazole-4-carboxylic acid [5-chloro-2-(5-oxo-4,5-dihydro-[1,2,4]oxadiazol-3-yl)-phenyl]-amide;
5-Trifluoromethyl-2-(4-trifluoromethyl-phenyl)-oxazole-4-carboxylic acid [5-chloro-2-(1H-tetrazol-5-yl)-phenyl]-amide;
5-(4-Chloro-phenyl)-2-trifluoromethyl-furan-3-carboxylic acid [5-chloro-2-(5-oxo-4,5-dihydro-[1,2,4]oxadiazol-3-yl)-phenyl]-amide;
5-(4-Chloro-phenyl)-2-trifluoromethyl-furan-3-carboxylic acid [4,5-dichloro-2-(1H-tetrazol-5-yl)-phenyl]-amide;
5-(4-Chloro-phenyl)-2-methyl-furan-3-carboxylic acid [5-chloro-2-(5-oxo-4,5-dihydro-[1,2,4]oxadiazol-3-yl)-phenyl]-amide;
2-(4-Chloro-benzyl)-thiazole-4-carboxylic acid [5-chloro-2-(5-oxo-2,5-dihydro-[1,2,4]oxadiazol-3-yl)-phenyl]-amide;
1-(4-Bromo-benzyl)-5-methyl-1H-[1,2,3]triazole-4-carboxylic acid [5-chloro-2-(5-oxo-4,5-dihydro-[1,2,4]oxadiazol-3-yl)-phenyl]-amide;
4-Methyl-2-morpholin-4-yl-thiazole-5-carboxylic acid [5-chloro-2-(5-oxo-4,5-dihydro-[1,2,4]oxadiazol-3-yl)-phenyl]-amide;
4-Methyl-2-morpholin-4-yl-thiazole-5-carboxylic acid [5-chloro-2-(1H-tetrazol-5-yl)-phenyl]-amide;
2-(3-Chloro-phenyl)-5-methyl-2H-[1,2,3]triazole-4-carboxylic acid [5-chloro-2-(5-oxo-4,5-dihydro-[1,2,4]oxadiazol-3-yl)-phenyl]-amide;
4-{5-[5-Chloro-2-(5-oxo-4,5-dihydro-[1,2,4]oxadiazol-3-yl)-phenylcarbamoyl]-4-methyl-thiazol-2-yl}-piperidine-1-carboxylic acid tert-butyl ester;
4-Methyl-2-(4-trifluoromethyl-phenyl)-thiazole-5-carboxylic acid [5-chloro-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-4-morpholin-4-yl-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-4-dimethylamino-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-2-(5-oxo-4,5-dihydro-[1,2,4]oxadiazol-3-yl)-4-piperidin-1-yl-phenyl]-amide;
2-(3-Chloro-phenyl)-5-methyl-2H-[1,2,3]triazole-4-carboxylic acid [5-chloro-2-(1H-tetrazol-5-yl)-phenyl]-amide;
4-Methyl-2-pyridin-4-yl-thiazole-5-carboxylic acid [5-chloro-2-(1H-tetrazol-5-yl)-phenyl]-amide;
4-Methyl-2-(4-trifluoromethyl-phenyl)-thiazole-5-carboxylic acid [5-chloro-2-(5-oxo-4,5-dihydro-[1,2,4]oxadiazol-3-yl)-4-piperidin-1-yl-phenyl]-amide;
5-(Cyclopentanecarbonyl-amino)-3-methyl-thiophene-2-carboxylic acid [5-chloro-2-(1H-tetrazol-5-yl)-phenyl]-amide; or
2,4-Dichloro-6-[(4-methyl-2-morpholin-4-yl-thiazole-5-carbonyl)-amino]-benzoic acid;

or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkenyl group designates a straight or branched carbon chain containing one or more double bonds, including di-enes, tri-enes and poly-enes. In a preferred embodiment the alkenyl group of the invention comprises of from two to eight carbon atoms (C₂₋₈-alkenyl), more preferred of from two to six carbon atoms (C₂₋₆-alkenyl), including at least one double bond. In a most preferred embodiment the alkenyl group of the invention is ethenyl; 1- or 2-propenyl (allyl); 1-, 2- or 3-butenyl, or 1,3-butadienyl; 1-, 2-, 3-, 4- or 5-hexenyl, or 1,3-hexadienyl, or 1,3,5-hexatrienyl; 1-, 2-, 3-, 4-, 5-, 6-, or 7-octenyl, or 1,3-octadienyl, or 1,3,5-octatrienyl, or 1,3,5,7-octatetraenyl.

In the context of this invention an alkynyl group designates a straight or branched carbon chain containing one or more triple bonds, including di-ynes, tri-ynes and poly-ynes. In a preferred embodiment the alkynyl group of the invention comprises of from two to eight carbon atoms (C₂₋₈-alkynyl), more preferred of from two to six carbon atoms (C₂₋₈-alkynyl), including at least one triple bond. In its most preferred embodiment the alkynyl group of the invention is ethynyl; 1-, or 2-propynyl; 1-, 2-, or 3-butynyl, or 1,3-butadiynyl; 1-, 2-, 3-, 4-pentynyl, or 1,3-pentadiynyl; 1-, 2-, 3-, 4-, or 5-hexynyl, or 1,3-hexadiynyl or 1,3,5-hexatrienyl; 1-, 2-, 3-, 4-, 5- or 6-heptynyl, or 1,3-heptadiynyl, or 1,3,5-heptatriynyl; 1-, 2-, 3-, 4-, 5-, 6- or 7-octynyl, or 1,3-octadiynyl, or 1,3,5-octatriynyl, or 1,3,5,7-octatetraynyl.

In the context of this invention an N-alkyl-amino group designates a (secondary) amino group, monosubstituted with an alkyl group as defined above.

In the context of this invention an N-aryl-amino group designates a (secondary) amino group, monosubstituted with an aryl group as defined below.

In the context of this invention an N,N-dialkyl-amino group designates a (tertiary) amino group, disubstituted with alkyl groups as defined above.

In the context of this invention an N,N-diaryl-amino group designates a (tertiary) amino group, disubstituted with aryl groups as defined above.

In the context of this invention an alkyl-sulfonyl-amino group designates an “alkyl-SO₂—NH—” group, wherein alkyl is as defined above.

In the context of this invention an aryl-sulfonyl-amino group designates an “aryl-SO₂—NH—” group, wherein aryl is as defined below.

In the context of this invention an alkyl-carbonyl-amino group designates an “alkyl-CO—NH—” group, wherein alkyl is as defined above.

In the context of this invention an aryl-carbonyl-amino group designates an “aryl-CO—NH—” group, wherein aryl is as defined below.

In the context of this invention alkoxy-carbonyl designates an “alkoxy-CO—” group, wherein alkoxy is as defined above.

In the context of this invention halo represents fluoro, chloro, bromo or iodo.

In the context of this invention an aryl group designates a monocyclic or polycyclic aromatic hydrocarbon group. Examples of preferred aryl groups of the invention include phenyl, indenyl, naphthyl, azulenyl, fluorenyl, and anthracenyl. In a most preferred embodiment an aryl group of the invention is phenyl.

### Pharmaceutically Acceptable Salts

The aromatic heterocyclic carboxylic acid amide derivatives of the invention may be provided in any form suitable for the intended administration. Suitable forms include pharmaceutically (i.e. physiologically) acceptable salts, and pre- or prodrug forms of the aromatic heterocyclic carboxylic acid amide derivative of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of an aromatic heterocyclic carboxylic acid amide derivative of the invention include, without limitation, the sodium, the potassium, the calcium, the magnesium, the lithium, and the ammonium salt, and the like, of an aromatic heterocyclic carboxylic acid amide derivative of the invention containing an anionic group. Such cationic salts may be formed by procedures well known and described in the art.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Preparation

The compounds according to the invention may be prepared by conventional methods for chemical synthesis, e.g. those described in the working examples.

### Biological Activity

The aromatic heterocyclic carboxylic acid amide derivatives of the invention have been found to possess potassium channel modulating activity as measured by standard electrophysiological methods. Due to their activity at the potassium channels, the aromatic heterocyclic carboxylic acid amide derivatives of the invention are considered useful for the treatment of a wide range of diseases and conditions.

In a special embodiment, the aromatic heterocyclic carboxylic acid amide derivatives of the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.

In a more preferred embodiment, the aromatic heterocyclic carboxylic acid amide derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the aromatic heterocyclic carboxylic acid amide derivatives of the invention are considered useful for the treatment, prevention or alleviation of psychosis, schizophrenia, bipolar disorder, depression, epilepsy, Parkinson's disease or pain.

In a third more preferred embodiment, the aromatic heterocyclic carboxylic acid amide derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the aromatic heterocyclic carboxylic acid amide derivatives of the invention are considered useful for the treatment, prevention or alleviation of cardiac ischemia, ischemic heart disease, hypertrophic heart, cardiomyopathy or failing heart.

In a fifth more preferred embodiment, the compounds of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In an sixth more preferred embodiment, the compounds of the invention are considered useful for obtaining preconditioning of the heart. Preconditioning, which includes ischemic preconditioning and myocardial preconditioning, describes short periods of ischemic events before initiation of a long lasting ischemia. The compounds of the invention are believed having an effect similar to preconditioning obtained by such ischemic events. Preconditioning protects against later tissue damage resulting from the long lasting ischemic events.

In a seventh more preferred embodiment, the aromatic heterocyclic carboxylic acid amide derivatives of the invention are considered useful for the treatment, prevention or alleviation of schizophrenia, depression or Parkinson's disease.

In an eighth more preferred embodiment, the compounds of the invention are considered useful for the treatment, prevention or alleviation of an obstructive or inflammatory airway disease. In a more preferred embodiment the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In a ninth more preferred embodiment, the aromatic heterocyclic carboxylic acid amide derivatives of the invention are considered useful for the treatment, prevention or alleviation of a sexual dysfunction, incl. male sexual dysfunction and female sexual dysfunction, and incl. male erectile dysfunction.

In an even more preferred embodiment the aromatic heterocyclic carboxylic acid amide derivatives of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the aromatic heterocyclic carboxylic acid amide derivatives of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred aromatic heterocyclic carboxylic acid amide derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of an aromatic heterocyclic carboxylic acid amide derivative of the invention.

While an aromatic heterocyclic carboxylic acid amide derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the aromatic heterocyclic carboxylic acid amide derivative of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) an aromatic heterocyclic carboxylic acid amide derivative of the invention; and

(B1) a phosphodiesterase inhibitor; or

(B2) an agent that potentiates endothelium-derived hyperpolarizing factor-mediated responses; and optionally

(C) instructions for the simultaneous, sequential or separate administration of the aromatic heterocyclic carboxylic acid amide derivative of A, and the phosphodiesterase inhibitor of B1, or an agent that potentiates endothelium-derived hyperpolarizing factor-mediated responses of B2, to a patient in need thereof.

In a more preferred embodiment the phosphodiesterase inhibitor for use according to the invention (B1) is a phosphodiesterase 5 (PDE5) inhibitor, and in an even more preferred embodiment the phosphodiesterase inhibitor for use according to the invention is sildenafil, tadalafil or vardenafil.

In another more preferred embodiment the agent that potentiates endothelium-derived hyperpolarizing factor-mediated responses for use according to the invention (B2) is calcium dobesilate.

The aromatic heterocyclic carboxylic acid amide derivative of the invention and the phosphodiesterase inhibitor or the agent that potentiates endothelium-derived hyperpolarizing factor-mediated responses for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the aromatic heterocyclic carboxylic acid amide derivative of the invention and the phosphodiesterase inhibitor or the agent that potentiates endothelium-derived hyperpolarizing factor-mediated responses for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the positive allosteric nicotine receptor modulator and the cognitive enhancer are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides a method of treatment, prevention or alleviation of a disease, disorder or condition of a living animal body, including a human, which disorder, disease or condition is responsive to activation of a potassium channel, which method comprises the step of administering to such a living animal body in need thereof, a therapeutically effective amount a compound capable of activating the potassium channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWINGS

The present invention is further illustrated by reference to the accompanying drawing, in which FIGS. 1A and 1B show the effect of Compound 2 (i.e. 1-(4-Chloro-phenyl)-5-trifluoromethyl-1H-pyrazole-4-carboxylic acid [5-chloro-2-(1H-tetrazol-5-yl)-phenyl]amide), FIGS. 2A and 2B show the effect of Compound 1 (i.e. 5-(4-Chloro-phenyl)-2-trifluoromethyl-furan-3-carboxylic acid [5-chloro-2-(1H-tetrazol-5-yl)-phenyl]-amide) and FIGS. 3A and 3B show the effect of Compound 9 (i.e. 4-Methyl-2-(4-trifluoromethyl-phenyl)-thiazole-5-carboxylic acid [5-chloro-2-(1H-tetrazol-5-yl)-phenyl]-amide) on the voltage dependence of BK_{Ca}, channels expressed in Xenopus Oocytes:

FIG. 1A shows conductance (μS) vs. membrane potential (mV) in the absence (Control) of Compound 2 and in the presence of 0.01 to 31.6 μM of Compound 2;

FIG. 1B shows the concentration-response relationship for the left-shift of the BK_{ca}-activation curve induced by Compound 2; i.e. ΔV (mV) vs. log [c] (M). The calculated EC50-value is 2.4 μM and the maximal left-shift for the BK-activation curve is −94 mV.

FIG. 2A shows conductance (μS) vs. membrane potential (mV) in the absence (Control) of Compound 1 and in the presence of 0.01 to 31.6 μM of Compound 1;

FIG. 2B shows the concentration-response relationship for the left-shift of the BK_{Ca}-activation curve induced by Compound 1; i.e. ΔV (mV) vs. log [c] (M). The calculated EC50-value is 4.4 μM and the maximal left-shift for the BK-activation curve is −88 mV.

FIG. 3A shows conductance (μS) vs. membrane potential (mV) in the absence (Control) of Compound 9 and in the presence of 0.01 to 31.6 μM of Compound 9;

FIG. 3B shows the concentration-response relationship for the left-shift of the BK_{ca}-activation curve induced by Compound 9; i.e. ΔV (mV) vs. log [c] (M). The calculated EC50-value is 0.97 μM and the maximal left-shift for the BK-activation curve is −99 mV.

### EXAMPLES

The invention is further illustrated with reference to the following examples, which are not intended to be in any way limiting to the scope of the invention as claimed.

### Example 1

### Preparatory Example

Abbreviations used herein:

AcOEt: ethyl acetate

CFM: chloroform

DCM: dichloromethane

DMF: N,N-dimethylformamide

DMAP: 4-dimethylaminopyridine

EDC.HCl: 1-ethyl-3-(3-dimethylaminopropyl)carbodiimide hydrochloride

EtOH: ethanol

Hex: hexane

MeOH: methanol

MgSO4: magnesium sulphate

PE: petroleum ether (fraction boiling at 40-60° C.)

Py: pyridine

TEA: triethylamine

THF: tetrahydrofuran

TOL: toluene

### Preparation of Intermediates

### N-(4-Chloro-2-nitro-benzoyl)-methanesulfonamide (INT-1) (Scheme 1)

To a suspension of 4-chloro-2-nitrobenzoic acid (5 g, 1 eq) in DCM (100 ml), EDC.HCl (9.5 g, 2 eq) and DMAP (9 g, 3 eq) are added. The resulting brown solution is stirred for 10 min and methanesulfonamide (2.35 g, 1 eq) is then added. The reddish solution is stirred at room temperature overnight, diluted with DCM (100 ml), washed with 1.5N HCl (2×50 ml) and water (50 ml), dried and evaporated to dryness, to give a yellowish solid (4.6 g, 67% yield). This crude material is suspended in DCM (15 ml), stirred for 15 min, filtered and dried, to afford the title compound as white solid (2 g, 29%, purity 98%), which is used as such for the next step.

### N-(2-Amino-4-chloro-benzoyl)-methanesulfonamide (INT-2) (Scheme 1)

To a solution of the intermediate H (1 g, 1 eq) in MeOH (80 ml), Raney-nickel (0.2 g) is carefully added. The reaction mixture is hydrogenated for 7 hours and then filtered through celite. The celite is washed with MeOH (50 ml) and the filtrate is evaporated to give the title compound as an off-white solid (0.8 g, 90% yield; 84% purity). The compound, as such, is used for the next step without further purification.

### (5-Chloro-2-hydroxy-phenyl)-carbamic acid tert-butyl ester (INT-3) (Scheme 2)

To an ice-cooled solution of commercial 2-amino-4-chlorophenol (10 g, 1 eq) in 1,4-dioxane (50 ml), an ice-cooled solution of boc-anhydride (15.96 g, 1.05 eq) in 1,4-dioxane is added and the resulting mixture is stirred at room temperature overnight. The reaction mixture is then diluted with DCM (100 ml) and washed with water (2×100 ml), brine (2×100 ml), dried over MgSO₄ and evaporated to dryness, to give a brownish gummy material (17.2 g), which is purified by flash column chromatography (using 230-400 mesh silica gel) and eluting with 4% AcOEt in Hex (12.1 g, yield 71%).

### (5-Chloro-2-cyanomethoxy-phenyl)-carbamic acid tert-butyl ester (INT-4) (Scheme 2)

To a suspension of INT-3 (5 g, 1 eq) in 2-butanone (20 ml), sodium iodide (3.69 g, 1.2 eq) and potassium carbonate (3.40 g, 1.2 eq) and chloroacetonitrile (1.86 g, 1.2 eq) are added. The reaction mixture is heated at 85° C. for 24 hr and then poured into water (100 ml), extracted with AcOEt (2×75 ml). The organic phases collected together are washed with water (100 ml), brine (100 ml), dried over MgSO₄ and evaporated to dryness to give a brownish semi-solid (5.08 g, 100% yield).

### [5-Chloro-2-(1H-tetrazol-5-ylmethoxy)-phenyl]-carbamic acid tert-butyl ester (INT-5) (Scheme 2)

A suspension of INT-4 (0.2 g, 1 eq), sodium azide (0.092 g, 2 eq) and ammonium chloride (0.077, 2 eq) in DMF (5 ml) is heated (120° C.) for 6 hours. The reaction mixture is evaporated to dryness to give a brownish gummy residue, which is suspended in a mixture of water (50 ml) and 1.5N HCl (10 ml) and stirred for 30 min at room temperature. The white solid formed is filtered, washed with water and dried to give the title compound (0.200 mg, 86% yield).

### 5-Chloro-2-(1H-tetrazol-5-ylmethoxy)-phenylamine (INT-6) (Scheme 2)

To a stirred and ice-cooled suspension of INT-5 (0.5 g, 1 eq) in DCM (30 ml), trifluoroacetic acid (1.19 ml, 10 eq) in DCM (10 ml) is added drop wise and the resulting mixture is stirred at room temperature for 2 hours. The organic solution is washed several times with water and dried over MgSO₄, to provide the title compound (0.35 g, 100% yield).

INT-7, INT-8, INT-9 were synthesised as described by Ishikawa et al. in Journal of Medicinal Chemistry 1985 28 (10) 1387-1393.

### 2-Amino-4-chloro-N-hydroxy-5-morpholin-4-yl-benzamidine (INT-10) (Scheme 3)

To a stirred and boiling solution of INT-7 (0.200 g, 1 eq) in EtOH (10 ml), a solution of hydroxylamine hydrochloride (0.119 g, 2 eq) and sodium bicarbonate (0.212 g, 3 eq) in water (5 ml) is added and reflux is continued for 8 hours. The reaction mixture is evaporated to dryness and the residue is suspended in water (20 ml) and the resulting suspension is filtered, washed with water and dried (0.140 g, yield 63%). The compound, as such, is used for the next step.

### 2-Amino-4-chloro-5-dimethylamino-N-hydroxy-benzamidine (INT-11) (Scheme 3)

To a stirred and boiling solution of INT-8 (0.200 g, 1 eq) in EtOH (10 ml), a solution of hydroxylamine hydrochloride (0.145 g, 2 eq) and sodium bicarbonate (0.257 g, 3 eq) in water (5 ml) is added and reflux is continued for 8 hours. The reaction mixture is evaporated to dryness and the residue is suspended in water (20 ml) and the resulting suspension is filtered, washed with water and dried (0.150 g, yield 67%). The compound, as such, is used for the next step.

### 2-Amino-4-chloro-N-hydroxy-5-piperidin-1-yl-benzamidine (INT-12) (Scheme 3)

To a stirred and boiling solution of INT-9 (0.200 g, 1 eq) in EtOH (10 ml), a solution of hydroxylamine hydrochloride (0.120 g, 2 eq) and sodium bicarbonate (0.214 g, 3 eq) in water (5 ml) is added and reflux is continued for 8 hours. The reaction mixture is evaporated to dryness and the residue is suspended in water (20 ml) and the resulting suspension is filtered, washed with water and dried (0.190 g, yield 86%). The compound, as such, is used for the next step.

### 3-(2-Amino-4-chloro-5-morpholin-4-yl-phenyl)-4H-[1,2,4]oxadiazol-5-one (INT-13) (Scheme 3)

To a stirred and boiling solution of sodium (0.0256 g, 2 eq) in absolute EtOH (10 ml), INT-10 (0.150 g, 1 eq) is added and reflux is continued for 1 hour. 0.27 ml (4 eq) of diethyl carbonate are then added and reflux is continued for 24 hours. The reaction mixture is evaporated to dryness and the residue is suspended in HCl 0.5 M (5 ml). The suspension is stirred overnight and filtered, to afford the title compound as a white powder (0.120 g, yield 73%), which is used as such for the next step.

### 3-(2-Amino-4-chloro-5-dimethylamino-phenyl)-4H-[1,2,4]oxadiazol-5-one (INT-14) (Scheme 3)

To a stirred and boiling solution of sodium (0.0202 g, 2 eq) in absolute EtOH (10 ml), INT-11 (0.100 g, 1 eq) is added and reflux is continued for 1 hour. 0.214 ml (4 eq) of diethyl carbonate are then added and reflux is continued for 24 hours. The reaction mixture is evaporated to dryness and the residue is suspended in HCl 0.5 M (5 ml). The suspension is stirred overnight and filtered, to afford the title compound as a white powder (0.072 g, yield 63%), which is used as such for the next step.

### 3-(2-Amino-4-chloro-5-piperidin-1-yl-phenyl)-4H-[1,2,4]oxadiazol-5-one (INT-15) (Scheme 3)

To a stirred and boiling solution of sodium (0.507 g, 2 eq) in absolute EtOH (30 ml), INT-11 (2.95 g, 1 eq) is added and reflux is continued for 1 hour. 5.37 ml (4 eq) of diethyl carbonate are then added and reflux is continued for 24 hours. The reaction mixture is evaporated to dryness and the residue is suspended in HCl 0.5 M (25 ml). The suspension is stirred overnight and filtered, to afford the title compound as a white powder (0.072 g, yield 63%), which is used as such for the next step. M.p. 186.3-189.4° C.

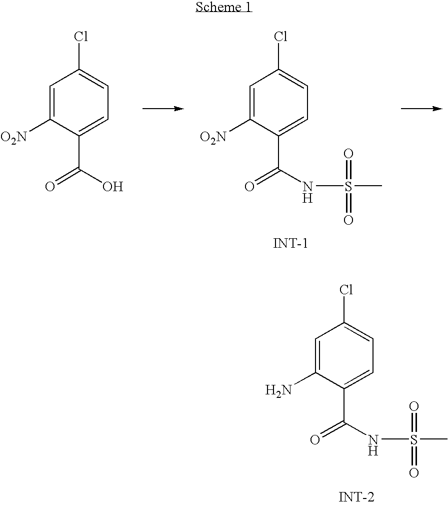

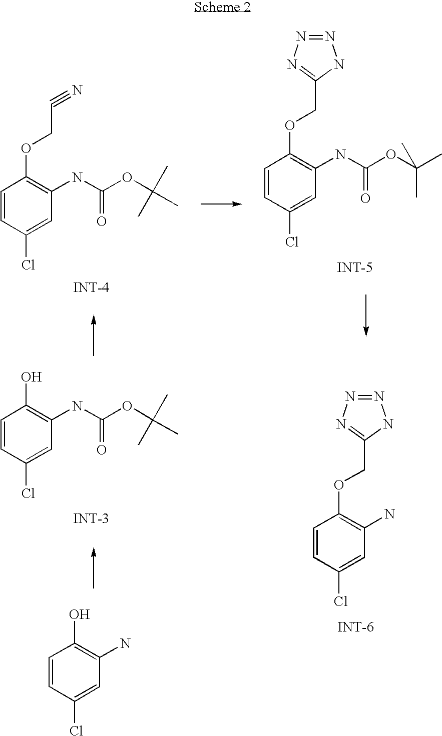

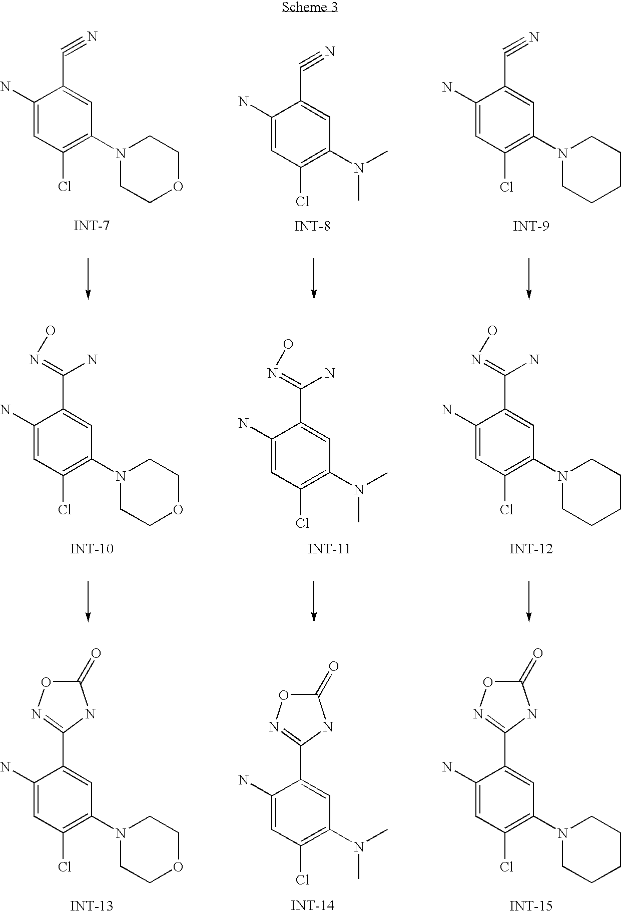

### 5-(4-Chloro-phenyl)-2-trifluoromethyl-furan-3-carboxylic acid [5-chloro-2-(1H-tetrazol-5-yl)-phenyl]-amide (1)

To a solution of the commercial 5-(4-chlorophenyl)-2-(trifluoromethyl)furan-3-carbonyl chloride (0.2 g, 1 eq) in dry THF (6 ml) and under nitrogen, TEA (0.45 ml, 5 eq) and 5-chloro-2-(1H-tetrazol-5-yl)-phenylamine (0.150 g, 1 eq) (prepared as described by Valgeirsson et al. in Journal of Medicinal Chemistry 2004 47 (27) 6948-6957) are added. The mixture is kept stirring under nitrogen and at room temperature overnight and it is then poured into water (20 ml). The resulting suspension is extracted with ethyl acetate (3×25 ml) and the organic phases collected together are washed with 1.5N HCl, water, dried over MgSO4 and evaporated to dryness, to get a yellow solid (0.32 g). This crude material is purified by flash column chromatography (using 230-400 mesh silica gel) and eluting with 8% MeOH in CFM, to afford the title compound as white powder (0.105 g, 32% yield). LC-ESI-HRMS of [M−H]− shows 466.0078 Da. Calc. 466.00854 Da, dev.-1.6 ppm.

### 1-(4-Chloro-phenyl)-5-trifluoromethyl-1H-pyrazole-4-carboxylic acid [5-chloro-2-(1H-tetrazol-5-yl)-phenyl]-amide (2)

To a suspension of commercial 2-(4-chlorophenyl)-3-(trifluoromethyl)-pyrazole-4-carboxylic acid (0.235 g, 1 eq) in DCM (10 ml), EDC.HCl (0.31 g, 2 eq) and DMAP (0.30 g, 3 eq) are added. The resulting mixture is stirred at room temperature for 10 min and 5-chloro-2-(1H-tetrazol-5-yl)-phenylamine (0.144 g, 0.9 eq) (prepared as described by Valgeirsson et al. in Journal of Medicinal Chemistry 2004 47 (27) 6948-6957) is added and stirring is continued at room temperature overnight. The reaction mixture is diluted with DCM (20 ml), washed with 1.5N HCl (2×15 ml) and water (15 ml), dried and evaporated to dryness to give a yellowish solid (0.255 g). This crude material is purified by flash chromatography using silica gel (230-400 mesh) and eluting with 0-5% MeOH in CFM, to afford the title compound as white powder (0.090 g, 24% yield). M.p. 176.3-180.4° C. LC-ESI-HRMS of [M−H]− shows 466.0193 Da. Calc. 466.019773 Da, dev.-1 ppm.

### 1-(4-Bromo-benzyl)-5-methyl-1H-[1,2,3]-triazole-4-carboxylic acid [5-chloro-2-(1H-tetrazol-5-yl)-phenyl]-amide (3)

To a stirred suspension of the commercial 1-(4-bromo-benzyl)-5-methyl-1H-[1,2,3]triazole-4-carboxylic acid (0.200 g, 1 eq) in DCM (15 ml), an excess of oxalyl chloride (1 ml) is added drop wise at 0° C., followed by 1-2 drops of dry DMF. The resulting yellow solution is allowed to reach room temperature spontaneously and then stirred at room temperature until starting material disappears completely on TLC (˜1 hour). The mixture is then evaporated to dryness under vacuum and the residue is taken up in DCM and washed with cold aqueous NaHCO₃. The organic phase, dried over MgSO₄ and evaporated to dryness, gives 0.212 g (˜100% yield) of 1-(4-bromo-benzyl)-5-methyl-1H-[1,2,3]triazole-4-carbonyl chloride, which is dissolved in TOL (10 ml) and Py (0.5 ml) under nitrogen. The new solution is ice-cooled and 5-chloro-2-(1H-tetrazol-5-yl)-phenylamine (0.119 g, 0.9 eq) (prepared as described by Valgeirsson at al. in Journal of Medicinal Chemistry 2004 47 (27) 6948-6957) is added and stirring is continued under nitrogen at room temperature overnight. The reaction mixture is evaporated to dryness and the crude solid residue is purified by crystallisation from MeOH (0.105 g, 22% yield). LC-ESI-HRMS of [M−H]− shows 471.007 Da. Calc. 471.008423 Da, dev.-3 ppm.

### 5-(4-Chloro-phenyl)-2-trifluoromethyl-furan-3-carboxylic acid (5-chloro-2-phenyl-carbamoyl-phenyl)-amide (4)

To a suspension of commercial 5-(4-chlorophenyl)-2-(trifluoromethyl)furan-3-carboxylic acid (0.25 g, 1 eq) in DCM (10 ml), EDC.HCl (0.33 g, 2 eq) and DMAP (0.315 g, 3 eq) are added. The resulting brown solution is stirred for 10 min and commercial 2-amino-4-chloro-N-phenyl-benzamide (0.21 g, 1 eq) is then added. The reaction mixture is stirred at room temperature overnight, diluted with DCM (20 ml), washed with 1.5N HCl (2×25 ml) and water (25 ml) and finally dried over MgSO₄ and evaporated to dryness, to give a yellowish solid (0.315 g). This crude material is purified by flash chromatography using silica gel (230-400 mesh) and eluting with 0-10% AcOEt in Hex, to afford the title compound as white powder (0.120 g, 27% yield). M.p. 176.3-180.4° C.

### 5-(4-Chloro-phenyl)-2-trifluoromethyl-furan-3-carboxylic acid (5-chloro-2-methanesulfonylaminocarbonyl-phenyl)-amide (5)

To an ice-cooled solution of the commercial 5-(4-chlorophenyl)-2-(trifluoromethyl)furan-3-carbonyl chloride (0.200 g, 1 eq) in dry THF (10 ml) and under nitrogen, Py (0.1 ml, 2 eq) and INT-2 (0.161 g, 1 eq) are added. The mixture is kept stirring under nitrogen and at room temperature overnight and it is then poured into water (20 ml). The resulting suspension is extracted with ethyl acetate (3×25 ml) and the organic phases collected together are washed with 1.5N HCl, water, dried over MgSO₄ and evaporated to dryness, to get a yellow solid (0.200 g). This crude material is purified by flash column chromatography (using 60-120 mesh silica gel) and eluting with 5% MeOH in CFM, to afford the title compound as white powder (0.055 g, 16% yield).M.p.: 259-262° C. LC-ESI-HRMS of [M+H]+ shows 520.9935 Da. Calc. 520.995259 Da, dev.-3.4 ppm.

### 4-Chloro-2-{[5-(4-chloro-phenyl)-2-trifluoromethyl-furan-3-carbonyl]-amino}-benzene-sulfonic acid (6)

To a suspension of commercial 5-(4-chlorophenyl)-2-(trifluoromethyl)furan-3-carboxylic acid (0.27 g, 1 eq) in DCM (15 ml), EDC.HCl (0.214 g, 2 eq) and DMAP (0.227 g, 2 eq) are added. The resulting brown solution is stirred for 10 min and commercial 2-amino-4-chloro-benzenesulfonic acid (0.202 g, 1 eq) prepared as described by Valgeirsson et al. in Journal of Medicinal Chemistry 2004 47 (27) is then added. The reaction mixture is stirred at room temperature overnight, diluted with DCM (20 ml), washed with 1.5N HCl (2×25 ml) and water (25 ml) and finally dried over MgSO₄ and evaporated to dryness, to give a yellowish solid (0.300 g). This crude material is purified by preparative HPLC (0.070 g, yield 16%), to afford the title compound as white powder. LC-ESI-HRMS of [M+H]+ shows 479.9685 Da. Calc. 479.96871 Da, dev.-0.4 ppm.

### 5-(4-Chloro-phenyl)-2-trifluoromethyl-furan-3-carboxylic acid [5-chloro-2-(1H-tetrazol-5-ylmethoxy)-phenyl]-amide (7)

To an ice-cooled solution of the commercial 5-(4-chlorophenyl)-2-(trifluoromethyl)furan-3-carbonyl chloride (0.500 g, 1 eq) in dry DCM (25 ml) and under nitrogen, TEA (0.64 ml, 3 eq) and INT-6 (0.346 g, 1 eq) are added. The mixture is kept stirring under nitrogen and at room temperature overnight and it is then evaporated to dryness to get an off-white solid (0.700 g). This crude residue is purified by flash chromatography on neutral alumina eluting with 20% MeOH in CFM, to afford the title compound as white powder (0.423 g, 55% yield). M.p. 226.9-236.1° C. LC-ESI-HRMS of [M+H]+ shows 498.0331 Da. Calc. 498.034755 Da, dev.-3.3 ppm.

### 4,5-Dichloro-2-{[5-(4-chloro-phenyl)-2-trifluoromethyl-furan-3-carbonyl]-amino}-benzenesulfonic acid (8)

To an ice-cooled solution of the commercial 5-(4-chlorophenyl)-2-(trifluoromethyl)furan-3-carbonyl chloride (0.250 g, 1 eq) in dry DCM (15 ml) and under nitrogen, Py (0.2 ml, 3 eq) and 2-amino-4,5-dichloro-benzenesulfonic acid (0.125 g, 1 eq) (prepared as described in DE 4112692) are added. The mixture is kept stirring under nitrogen and at room temperature overnight and then diluted with DCM (20 ml). The new solution is washed with 1.5N HCl (2×25 ml), water (2×25), dried over MgSO₄ and evaporated to dryness, to get a yellow solid (0.372 g). This crude material is purified by flash column chromatography (using 230-400 mesh silica gel) and eluting with 3% MeOH in CFM, to afford the title compound as white powder (0.145 g, 38% yield). LC-ESI-HRMS of [M+H]+ shows 513.9299 Da. Calc. 513.929738 Da, dev. 0.3 ppm.

### 4-Methyl-2-(4-trifluoromethyl-phenyl)-thiazole-5-carboxylic acid [5-chloro-2-(1H-tetrazol-5-yl)-phenyl]-amide (9)

To a solution of the commercial 4-methyl-2-[4-(trifluoromethyl)phenyl]-1,3-thiazole-5-carbonyl Chloride (0.300 g, 1 eq) in dry THF (7 ml) and under nitrogen, TEA (0.27 ml, 2 eq) and 5-chloro-2-(1H-tetrazol-5-yl)-phenylamine (0.194 g, 1 eq) (prepared as described by Valgeirsson et al. in Journal of Medicinal Chemistry 2004 47 (27) 6948-6957) are added. The mixture is kept stirring under nitrogen and at room temperature overnight and it is then poured into water (40 ml). The resulting suspension is extracted with ethyl acetate (3×40 ml) and the organic phases collected together are washed with 1.5N HCl, water, dried over MgSO4 and evaporated to dryness, to get a brown solid (0.325 g). This crude material is purified by flash column chromatography (using 230-400 mesh silica gel) and eluting with 2% MeOH in CFM, to afford the title compound as white powder (0.124 g, 27% yield). M.p. 325-337.2° C. LC-ESI-HRMS of [M+H]+ shows 465.0503 Da. Calc. 465.051217 Da, dev.-2 ppm.

### 4-Chloro-2-{[5-(4-chloro-phenyl)-2-trifluoromethyl-furan-3-carbonyl]-amino}-benzoic acid (10)

To a solution of the commercial 5-(4-chlorophenyl)-2-(trifluoromethyl)furan-3-carbonyl chloride (0.200 g, 1 eq) in dry DCM (10 ml) and under nitrogen, Py (0.13 ml, 3 eq) and commercial 2-amino-4-chlorobenzoic acid (0.111, 1 eq) are added. The mixture is kept stirring under nitrogen and at room temperature overnight. The resulting mixture is quenched with 1.5N HCl (20 ml), and then extracted with DCM (3 25×25 ml). The organic phases collected together are dried over MgSO4 and evaporated to dryness, to get a yellow solid (0.279 g). This crude material is purified by flash column chromatography (using 60-120 mesh silica gel) and eluting with 3% MeOH in CFM, to afford the title compound as white powder (0.191 g, 66% yield). LC-ESI-HRMS of [M−H]− shows 441.9844 Da. Calc. 441.986074 Da, dev.-3.8 ppm.

### 4-Chloro-2-{[5-(4-chloro-phenyl)-2-trifluoromethyl-furan-3-carbonyl]-amino}-benzoylcyanamide (11)

To an ice-cooled and stirred suspension of sodium hydride (0.027 g, 2 eq) in dry THF (5 ml), cyanamide (0.014 g, 1 eq) is added and stirring is continued for 30 min at room temperature. Compound 10, upon treatment with oxalyl chloride, is meanwhile converted to the corresponding acid chloride (4-chloro-2-{([5-(4-chloro-phenyl)-2-trifluoromethyl-furan-3-carbonyl]-amino}-benzoyl chloride). A solution of this latter (1 eq) in THF (15 ml) is added dropwise to the ice-cooled mixture of sodium cyanamide in THF, and stirring is continued for 1 h at room temperature. The reaction mixture is then quenched with 10 ml of water and extracted with AcOEt (3×30 ml). The organic layer is washed with water, dried over MgSO4 and evaporated to dryness to get a brownish solid (0.02 g), which is purified by flash column chromatography (using 60-120 mesh silica gel) and eluting with 8% MeOH in CFM, to afford the title compound as white powder (0.045 g, 25% yield). LC-ESI-HRMS of [M+H]+ shows 468.0115 Da. Calc. 468.012957 Da, dev.-3.1 ppm.

### 5-(4-Chloro-phenyl)-2-methyl-furan-3-carboxylic acid [5-chloro-2-(1H-tetrazol-5-yl)-phenyl]-amide (12)

To a solution of the commercial 5-(4-chlorophenyl)-2-methylfuran-3-carbonyl chloride (0.100 g, 1 eq) in dry THF (10 ml) and under nitrogen, TEA (0.08 ml, 1.5 eq) and 5-chloro-2-(1H-tetrazol-5-yl)-phenylamine (0.078 g, 1 eq) (prepared as described by Valgeirsson et al. in Journal of Medicinal Chemistry 2004 47 (27) 6948-6957) are added. The mixture is kept stirring under nitrogen and at room temperature overnight. The resulting mixture is quenched with 1.5N HCl (10 ml), and then extracted with AcOEt (3×25 ml). The organic phases collected together are dried over MgSO4 and evaporated to dryness, to get a white solid (0.135 g). This crude material is purified by crystallization from AcOEt/PE, to afford the title compound as an off-white solid (0.042 g, 26% yield). M.p. 246.7-255.4° C. LC-ESI-HRMS of [M+H]+ shows 414.0537 Da. Calc. 414.052456 Da, dev. 3 ppm.

### 2-{[5-(4-Chloro-phenyl)-2-trifluoromethyl-furan-3-carbonyl]-amino}-4,5-difluoro-benzoic acid (13)

To a solution of the commercial 5-(4-chlorophenyl)-2-(trifluoromethyl)furan-3-carbonyl chloride (0.100 g, 1 eq) in dry THF (15 ml) and under nitrogen, TEA (0.18 ml, 4 eq) and commercial 2-amino-4,5-difluorobenzoic acid (0.056, 1 eq) are added. The mixture is kept stirring under nitrogen and at room temperature overnight. The resulting mixture is quenched with 1.5N HCl (10 ml), and then extracted with AcOEt (3×25 ml). The organic phases collected together are dried over MgSO4 and evaporated to dryness, to get a white solid (0.122 g). This crude material is purified by flash column chromatography (using 60-120 mesh silica gel) and eluting with 3% MeOH in CFM, to afford the title compound as white powder (0.060 g, 42% yield). M.p. 337.2-340.2° C. LC-ESI-HRMS of [M−H]− shows 444.0058 Da. Calc. 444.006202 Da, dev.-0.9 ppm.

### 4,5-Dichloro-2-{[5-(4-chloro-phenyl)-2-trifluoromethyl-furan-3-carbonyl]-amino}-benzenesulfonic acid methyl ester (14)

To an ice-cooled suspension of 8 (0.070 g) in dry DCM (2 ml) methyl trifluoromethanesulfonate (0.025 g, 1.1 eq) and TEA (0.021 ml, 1.1 eq) are added and the resulting mixture is stirred at room temperature overnight. Evaporation to dryness provided ˜70 mg of crude solid material, which are purified by flash chromatography on neutral alumina and by elution with 4% AcOEt in PE (0.018 g, 25% yield). LC-ESI-HRMS of [M−H]− shows 525.9299 Da. Calc. 525.929738 Da, dev. 0.3 ppm.

### 5-Methyl-2-phenyl-2H-[1, 2, 3]triazole-4-carboxylic acid [5-chloro-2-(1H-tetrazol-5-yl)-phenyl]-amide (15)

To a suspension of 4-methyl-2-phenyl-1,2,3-triazole-5-carboxylic acid (0.25 g, 1 eq) in DCM (15 ml), EDC.HCl (0.472 g, 2 eq) and DMAP (0.451 g, 3 eq) are added. The resulting brown solution is stirred for 10 min and 5-chloro-2-(1H-tetrazol-5-yl)-phenylamine (0.241 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a yellowish solid (0.369 g). This crude material is purified by crystallisation from DCM (0.250, 53% yield). LC-ESI-HRMS of [M+H]+ shows 381.099 Da. Calc. 381.09791 Da, dev. 2.9 ppm.

### 5-Trifluoromethyl-2-(4-trifluoromethyl-phenyl)-oxazole-4-carboxylic acid [5-chloro-2-(5-oxo-4,5-dihydro-[1,2,4]oxadiazol-3-yl)-phenyl]-amide (16)

To a suspension of 5-(trifluoromethyl)-2-[4-(trifluoromethyl)phenyl]-1,3-oxazole-4-carboxylic acid (0.25 g, 1 eq) in DCM (15 ml), EDC.HCl (0.295 g, 2 eq) and DMAP (0.282 g, 3 eq) are added. The resulting solution is stirred for 10 min and 3-(2-amino-4-chloro-phenyl)-4H-[1,2,4]oxadiazol-5-one (0.166 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a pure white solid (0.176 g, 45% yield). LC-ESI-HRMS of [M−H]−shows 517.0135 Da. Calc. 517.013827 Da, dev.-0.6 ppm.

### 5-Trifluoromethyl-2-(4-trifluoromethyl-phenyl)-oxazole-4-carboxylic acid [5-chloro-2-(1H-tetrazol-5-yl)-phenyl]-amide (17)

To a suspension of 5-(trifluoromethyl)-2-[4-(trifluoromethyl)phenyl]-1,3-oxazole-4-carboxylic acid (0.25 g, 1 eq) in DCM (15 ml), EDC.HCl (0.295 g, 2 eq) and DMAP (0.282 g, 3 eq) are added. The resulting solution is stirred for 10 min and 5-chloro-2-(1H-tetrazol-5-yl)-phenylamine (0.150 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (0.340 g) that is purified by crystallisation from EtOH (0.142 g, 37%). LC-ESI-HRMS of [M−H]− shows 501.0297 Da. Calc. 501.030145 Da, dev.-0.9 ppm.

### 5-(4-Chloro-phenyl)-2-trifluoromethyl-furan-3-carboxylic acid [5-chloro-2-(5-oxo-4,5-dihydro-[1,2,4]oxadiazol-3-yl)-phenyl]-amide (18)

To a solution of the commercial 5-(4-chlorophenyl)-2-(trifluoromethyl)furan-3-carbonyl chloride (0.2 g, 1 eq) in dry THF (6 ml) and under nitrogen, TEA (0.45 ml, 5 eq) and 3-(2-amino-4-chloro-phenyl)-4H-[1,2,4]oxadiazol-5-one (0.160 g, 1 eq) (prepared as described by Valgeirsson et al. in Journal of Medicinal Chemistry 2004 47 (27) 6948-6957) is added. The mixture is kept stirring under nitrogen and at room temperature overnight and it is then poured into water (20 ml). The resulting suspension is extracted with ethyl acetate (3×25 ml) and the organic phases collected together are washed with 1.5N HCl, water, dried over MgSO4 and evaporated to dryness, to get a yellow solid (0.301 g). This crude material is purified by flash column chromatography (using 230-400 mesh silica gel) and eluting with 20% AcOEt in Hex, to afford the title compound as white powder (0.145 g, 36% yield). LC-ESI-HRMS of [M+H]+ shows 484.0063 Da. Calc. 484.007872 Da, dev.-3.2 ppm.

### 5-(4-Chloro-phenyl)-2-trifluoromethyl-furan-3-carboxylic acid [4,5-dichloro-2-(1H-tetrazol-5-yl)-phenyl]-amide (19)

To a solution of the commercial 5-(4-chlorophenyl)-2-(trifluoromethyl)furan-3-carbonyl chloride (0.1 g, 1 eq) in dry DCM (6 ml) and under nitrogen, TEA (0.225 ml, 5 eq) and 4,5-dichloro-2-(1H-tetrazol-5-yl)-phenylamine (0.074, 1 eq) is added, as described in e.g. U.S. Pat. No. 3,838,126. The resulting mixture is quenched with 1.5N HCl (10 ml), and then extracted with DCM (3×20 ml). The organic phases collected together are dried over MgSO4 and evaporated to dryness, to get a yellow solid (0.160 g). This crude material is purified by flash column chromatography (using 60-120 mesh silica gel) and eluting with 5% MeOH in CFM, to afford the title compound as white powder (0.030 g, 18% yield). LC-ESI-HRMS of [M−H]− shows 499.9698 Da. Calc. 499.969568 Da, dev. 0.5 ppm.

### 5-(4-Chloro-phenyl)-2-methyl-furan-3-carboxylic acid [5-chloro-2-(5-oxo-4,5-dihydro-[1,2,4]oxadiazol-3-yl)-phenyl]-amide (20)

To a solution of the commercial 5-(4-chloro-phenyl)-2-methyl-furan-3-carbonyl chloride (0.100 g, 1 eq) in dry THF (7 ml) and under nitrogen, TEA (˜0.3 ml, 5 eq) and 3-(2-Amino-4-chloro-phenyl)-4H-[1,2,4]oxadiazol-5-one (0.1167, 1.2 eq) (prepared as described by Valgeirsson et al. in Journal of Medicinal Chemistry 2004 47 (27) 6948-6957) are added. The resulting mixture is quenched with 1.5N HCl (10 ml), and then extracted with DCM (3×20 ml). The organic phases collected together are dried over MgSO4 and evaporated to dryness, to get a yellow solid (0.150 g). This crude material is purified by flash column chromatography (using 230-400 mesh silica gel) and eluting with 2% MeOH in CFM, to afford the title compound as white powder (0.055 g, 25% yield). LC-ESI-HRMS of [M−H]− shows 428.0211. Da. Calc. 428.020488 Da, dev. 1.4 ppm. M.p. 248.1-250.9° C.

### 2-(4-Chloro-benzyl)-thiazole-4-carboxylic acid [5-chloro-2-(5-oxo-2,5-dihydro-[1,2,4]oxadiazol-3-yl)-phenyl]-amide (21)

To a suspension of commercial 2-(4-chloro-benzyl)-thiazole-4-carboxylic acid (0.25 g, 1 eq) in DCM (10 ml), EDC.HCl (0.3778 g, 2 eq) and DMAP (0.3612 g, 3 eq) are added. The resulting solution is stirred for 10 min and 3-(2-amino-4-chloro-phenyl)-4H-[1,2,4]oxadiazol-5-one (0.2085 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (0.400 g) that is purified by preparative LCMS, to afford the title compound as white powder (0.088 g, 20% yield). LC-ESI-HRMS of [M+H]+ shows 447.008 Da. Calc. 447.008543 Da, dev.-1.2 ppm.

### 1-(4-Bromo-benzyl)-5-methyl-1H-[1,2,3]triazole-4-carboxylic acid [5-chloro-2-(5-oxo-4,5-dihydro-[1,2,4]oxadiazol-3-yl)-phenyl]-amide (22)

To a suspension of commercial 1-(4-bromo-benzyl)-5-methyl-1H-[1,2,3]triazole-4-carboxylic acid (0.25 g, 1 eq) in DCM (10 ml), EDC.HCl (0.3237 g, 2 eq) and DMAP (0.3094 g, 3 eq) are added. The resulting solution is stirred for 10 min and 3-(2-amino-4-chloro-phenyl)-4H-[1,2,4]oxadiazol-5-one (0.1786 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.300 g) that is purified by crystallisation from DMSO/water, to afford the title compound as white powder (˜0.080 g, ˜20% yield). LC-ESI-HRMS of [M+H]+ shows 489.0091 Da. Calc. 489.007755 Da, dev. 2.8 ppm.

### 4-Methyl-2-morpholin-4-yl-thiazole-5-carboxylic acid [5-chloro-2-(5-oxo-4,5-dihydro-[1,2,4]oxadiazol-3-yl)-phenyl]-amide (23)

To a suspension of commercial 4-methyl-2-morpholin-4-yl-thiazole-5-carboxylic acid (0.25 g, 1 eq) in DCM (10 ml), EDC.HCl (0.4538 g, 2 eq) and DMAP (0.4338 g, 3 eq) are added. The resulting solution is stirred for 10 min and 3-(2-amino-4-chloro-phenyl)-4H-[1,2,4]oxadiazol-5-one (0.250 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.370 g) that is purified by crystallisation from DMSO/water, to afford the title compound as white powder (0.126 g, ˜20% yield). LC-ESI-HRMS of [M+H]+ shows 422.0686 Da. Calc. 422.068979 Da, dev.-0.9 ppm.

### 4-Methyl-2-morpholin-4-yl-thiazole-5-carboxylic acid [5-chloro-2-(1H-tetrazol-5-yl)-phenyl]-amide (24)

To a suspension of commercial 4-methyl-2-morpholin-4-yl-thiazole-5-carboxylic acid (0.25 g, 1 eq) in DCM (10 ml), EDC.HCl (0.4538 g, 2 eq) and DMAP (0.4338 g, 3 eq) are added. The resulting solution is stirred for 10 min and 5-chloro-2-(1H-tetrazol-5-yl)-phenylamine (0.2315 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.390 g) that is purified by crystallisation from DMSO/water, to afford the title compound as white powder (0.240 g, ˜50% yield). LC-ESI-HRMS of [M+H]+ shows 406.0843 Da. Calc. 406.085297 Da, dev.-2.5 ppm.

### 2-(3-Chloro-phenyl)-5-methyl-2H-[1, 2, 3]triazole-4-carboxylic acid [5-chloro-2-(5-oxo-4,5-dihydro-[1,2,4]oxadiazol-3-yl)-phenyl]-amide (25)

To a suspension of commercial 2-(3-chloro-phenyl)-5-methyl-2H-[1,2,3]triazole-4-carboxylic acid (0.25 g, 1 eq) in DCM (10 ml), EDC.HCl (0.4033 g, 2 eq) and DMAP (0.3856 g, 3 eq) are added. The resulting solution is stirred for 10 min and 3-(2-amino-4-chloro-phenyl)-4H-[1,2,4]oxadiazol-5-one (0.2226 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.400 g) that is purified by crystallisation from methanol, to afford the title compound as white powder (0.302 g, ˜67% yield). LC-ESI-HRMS of [M−H]− shows 429.0278 Da. Calc. 429.02697 Da, dev. 1.9 ppm.

### 4-{5-[5-Chloro-2-(5-oxo-4,5-dihydro-[1,2,4]oxadiazol-3-yl)-phenylcarbamoyl ]-4-methyl-thiazol-2-yl}-piperidine-1-carboxylic acid tert-butyl ester (26)

To a suspension of commercial 4-(5-carboxy-4-methyl-thiazol-2-yl)-piperidine-1-carboxylic acid tert-butyl ester (0.25 g, 1 eq) in DCM (10 ml), EDC.HCl (0.2937 g, 2 eq) and DMAP (0.2807 g, 3 eq) are added. The resulting solution is stirred for 10 min and 3-(2-amino-4-chloro-phenyl)-4H-[1,2,4]oxadiazol-5-one (0.1621 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.370 g) that is purified by preparative LCMS, to afford the title compound as white powder (0.101 g, ˜25% yield). LC-ESI-HRMS of [M+H]+ shows 520.1411 Da. Calc. 520.142144 Da, dev.-2 ppm.

### 4-Methyl-2-(4-trifluoromethyl-phenyl)-thiazole-5-carboxylic acid [5-chloro-2-(5-oxo-4,5-dihydro-[1,2,4]oxadiazol-3-yl)-phenyl]-amide (27)

To a suspension of commercial 4-methyl-2-(4-trifluoromethyl-phenyl)-thiazole-5-carboxylic acid (1.00 g, 1 eq) in DCM (40 ml), EDC.HCl (0.8008 g, 1.2 eq) and DMAP (0.25104 g, 1.2 eq) are added. The resulting solution is stirred for 10 min and 3-(2-amino-4-chloro-phenyl)-4H-[1,2,4]oxadiazol-5-one (0.7366 g, 1 eq) (prepared as described by Valgeirsson et al. in Journal of Medicinal Chemistry 2004 47 (27) 6948-6957) is added. The solution is stirred at room temperature overnight, diluted with DCM (80 ml), washed with 1.5N HCl (120 ml) and water (120 ml), dried and evaporated to dryness, to give a solid residue (˜1.5 g) that is purified by flash column chromatography and eluting with 10% MeOH in DCM, to afford the title compound as a white powder (0.326 g, ˜20% yield). LC-ESI-HRMS of [M+H]+ shows 481.0338 Da. Calc. 481.034899 Da, dev.-2.3 ppm.

### 5-(4-Chloro-phenyl)-2-trifluoromethyl-furan-3-carboxylic acid [5-chloro-4-morpholin-4-yl-2-(5-oxo-4,5-dihydro-[1,2,4]oxadiazol-3-yl-phenyl]-amide (28)

To an ice-cooled solution of the commercial 5-(4-chlorophenyl)-2-(trifluoromethyl)furan-3-carbonyl chloride (0.500 g, 1 eq) in dry DCM (25 ml) and under nitrogen, TEA (0.67 ml, 3 eq) and INT-13 (0.48 g, 1 eq) are added. The mixture is kept stirring under nitrogen and at room temperature overnight and it is then evaporated to dryness to get an off-white solid (˜0.800 g). This crude residue is purified by flash chromatography eluting with 10% AcOEt in Hex, to afford the title compound as a white powder (0.131 g, 14% yield). M.p. 226.9-236.1° C. LC-ESI-HRMS of [M+H]+ shows 569.0627 Da. Calc. 569.060636 Da, dev. 3.6 ppm. M.p. 229-234° C.

### 5-(4-Chloro-phenyl)-2-trifluoromethyl-furan-3-carboxylic acid [5-chloro-4-dimethyl-amino-2-(5-oxo-4,5-dihydro-[1,2,4]oxadiazol-3-yl)-phenyl]-amide (29)

To an ice-cooled solution of the commercial 5-(4-chlorophenyl)-2-(trifluoromethyl)furan-3-carbonyl chloride (0.500 g, 1 eq) in dry DCM (25 ml) and under nitrogen, TEA (0.67 ml, 3 eq) and INT-14 (0.412 g, 1 eq) are added. The mixture is kept stirring under nitrogen and at room temperature overnight and it is then evaporated to dryness to get an off-white solid (˜0.600 g). This crude residue is purified by flash chromatography eluting with 10% AcOEt in Hex, to afford the title compound as a white powder (0.32 g, 37% yield). M.p. 256.2-258.4° C. LC-ESI-HRMS of [M+H]+ shows 527.0529 Da. Calc. 527.050071 Da, dev. 5.4 ppm.

### 5-(4-Chloro-phenyl)-2-trifluoromethyl-furan-3-carboxylic acid [5-chloro-2-(5-oxo-4,5-dihydro-[2,4]oxadiazol-3-yl)-4-piperidin-1-yl-phenyl]-amide (30)

To an ice-cooled solution of the commercial 5-(4-chlorophenyl)-2-(trifluoromethyl)furan-3-carbonyl chloride (0.400 g, 1 eq) in dry DCM (25 ml) and under nitrogen, TEA (0.54 ml, 3 eq) and INT-15 (0.381 g, 1 eq) are added. The mixture is kept stirring under nitrogen and at room temperature overnight and it is then evaporated to dryness to get an off-white solid (˜0.550 g). This crude residue is purified by flash chromatography eluting with 10% AcOEt in Hex, to afford the title compound as a white powder (0.312 g, 43% yield). M.p. 247.8-249.6° C. LC-ESI-HRMS of [M+H]+ shows 567.0822 Da. Calc. 567.081371 Da, dev. 1.5 ppm.

### 2-(3-Chloro-phenyl)-5-methyl-2H-[1, 2, 3]triazole-4-carboxylic acid [5-chloro-2-(1H-tetrazol-5-yl)-phenyl]-amide (31)

To a suspension of commercial 2-(3-chloro-phenyl)-5-methyl-2H-[1,2,3]triazole-4-carboxylic acid (0.25 g, 1 eq) in DCM (10 ml), EDC.HCl (0.4033 g, 2 eq) and DMAP (0.3856 g, 3 eq) are added. The resulting solution is stirred for 10 min and 5-chloro-2-(1H-tetrazol-5-yl)-phenylamine (0.2058 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.400 g) that is purified by crystallisation from methanol, to afford the title compound as white powder (0.190 g, ˜43% yield). LC-ESI-HRMS of [M+H]+ shows 415.0593 Da. Calc. 415.058938 Da, dev. 0.9 ppm.

### 4-Methyl-2-pyridin-4-yl-thiazole-5-carboxylic acid [5-chloro-2-(1H-tetrazol-5-yl)-phenyl]-amide (32)

To a suspension of commercial 4-methyl-2-pyridin-4-yl-thiazole-5-carboxylic acid (0.25 g, 1 eq) in DCM (10 ml), EDC.HCl (0.4352 g, 2 eq) and DMAP (0.416 g, 3 eq) are added. The resulting solution is stirred for 10 min and 5-chloro-2-(1H-tetrazol-5-yl)-phenylamine (0.2058 g, 1 eq) (prepared as described by Valgeirsson et al. in Journal of Medicinal Chemistry 2004 47 (27) 6948-6957) is added. The solution is stirred at room temperature overnight, diluted with DCM (20 ml), washed with 1.5N HCl (30 ml) and water (30 ml), dried and evaporated to dryness, to give a solid residue (˜0.410 g) that is purified by crystallisation from methanol, to afford the title compound as white powder (0.214 g, ˜47% yield). LC-ESI-HRMS of [M+H]+ shows 398.0612 Da. Calc. 398.059082 Da, dev. 5.3 ppm.

### 4-Methyl-2-(4-trifluoromethyl-phenyl)-thiazole-5-carboxylic acid [5-chloro-2-(5-oxo-4,5-dihydro-[1,2,4]oxadiazol-3-yl)-4-piperidin-1-yl-phenyl]-amide (33)

To a suspension of commercial 4-methyl-2-(4-trifluoromethyl-phenyl)-thiazole-5-carboxylic acid (0.500 g, 1 eq) in DCM (25 ml), EDC.HCl (0.667 g, 2 eq) and DMAP (0.638 g, 3 eq) are added. The resulting solution is stirred for 10 min and INT-15 (0.513 g, 1 eq) is added. The solution is stirred at room temperature overnight, diluted with DCM (30 ml), washed with 1.5N HCl (45 ml) and water (45 ml), dried and evaporated to dryness, to give a solid residue (˜0.720 g) that is purified by crystallisation from DMF/water (0.464 g, 47% yield). LC-ESI-HRMS of [M+H]+ shows 564.1078 Da. Calc. 564.108398 Da, dev.-1.1 ppm.

### 5-(Cyclopentanecarbonyl-amino)-3-methyl-thiophene-2-carboxylic acid [5-chloro-2-(1H-tetrazol-5-yl)-phenyl]-amide (34)

To a suspension of commercial 5-(cyclopentanecarbonyl-amino)-3-methyl-thiophene-2-carboxylic acid (0.500 g, 1 eq) in DCM (25 ml), EDC.HCl (0.454 g, 1.2 eq) and DMAP (0.2894 g, 1.2 eq) are added. The resulting solution is stirred for 10 min and 5-chloro-2-(1H-tetrazol-5-yl)-phenylamine (0.386 g, 1 eq) (prepared as described by Valgeirsson et al. in Journal of Medicinal Chemistry 2004 47 (27) 6948-6957) is added. The solution is stirred at room temperature overnight, diluted with DCM (30 ml), washed with 1.5N HCl (45 ml) and water (45 ml), dried and evaporated to dryness, to give a solid residue (0.750 g) that is purified by flash column chromatography and eluting with 10% MeOH in DCM, to afford the title compound as white powder (0.228 g, 26% yield). LC-ESI-HRMS of [M+H]+ shows 431.106 Da. Calc. 431.105698 Da, dev. 0.7 ppm.

### 2,4-Dichloro-6-[(4-methyl-2-morpholin-4-yl-thiazole-5-carbonyl)-amino]-benzoic acid (35)

To a stirred suspension of the commercial 4-Methyl-2-morpholin-4-yl-thiazole-5-carboxylic acid (0.4086 g, 1 eq) in DCM (30 ml), an excess of oxalyl chloride (1.5 ml) is added drop wise at 0° C., followed by 1-2 drops of dry DMF. The resulting yellow solution is allowed to reach room temperature spontaneously and then stirred at room temperature until starting material disappears completely on TLC (˜3 hours). The mixture is then evaporated to dryness under vacuum and the residue is taken up in DCM and washed with cold aqueous NaHCO₃. The organic phase, dried over MgSO₄ and evaporated to dryness, gives 0.440 g (˜100% yield) of 4-Methyl-2-morpholin-4-yl-thiazole-5-carbonyl chloride, which is dissolved in TOL (20 ml) and Py (1.5 ml) under nitrogen. The new solution is ice-cooled and 2-amino-4,6-dichloro-benzoic acid (0.295 g, 0.8 eq) (prepared as described by Sheibley et al. in Journal of Organic Chemistry 1956 21 (171-173) is added and stirring is continued under nitrogen at room temperature overnight. The reaction mixture is evaporated to dryness and the crude solid residue is purified by crystallisation from DMSO/water (0.256 g, 34% yield). LC-MS of [M−H]−: 414.

### Example 2

### Biological Activity

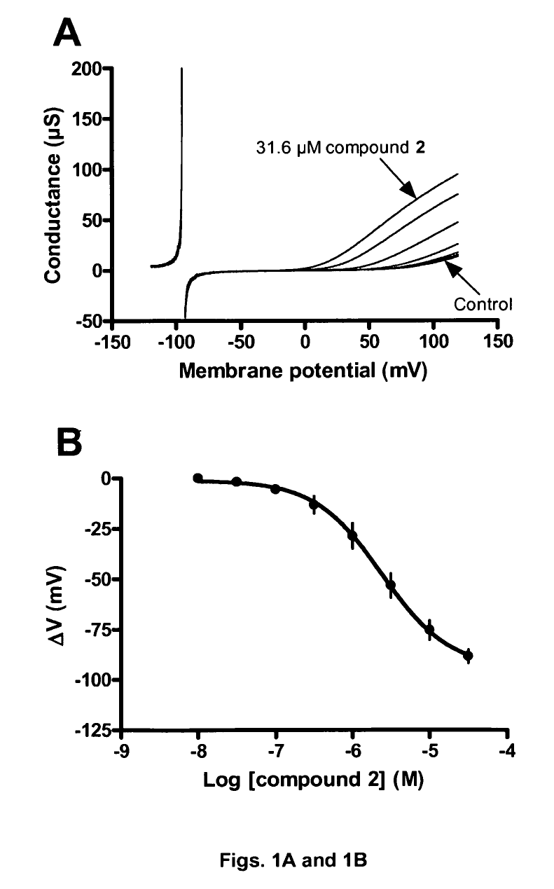

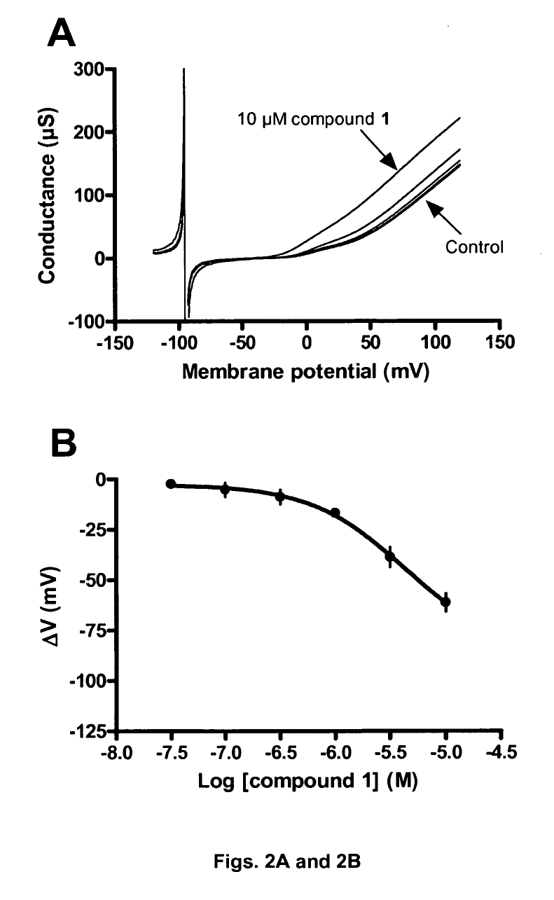

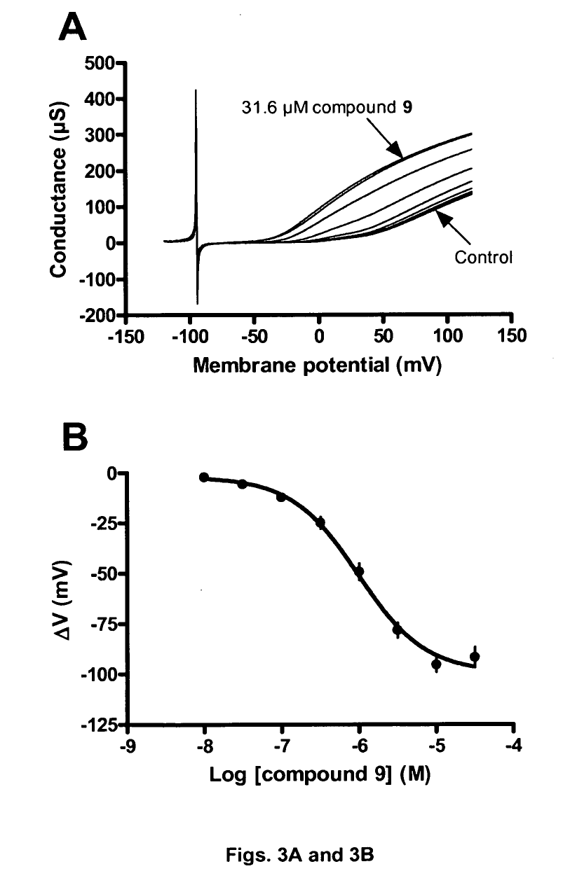

In this example the BK channel opening activity of Compound 2 (FIGS. 1A and 1B), Compound 1 (FIGS. 2A and 2B) and Compound 9 (FIGS. 3A and 3B) is determined using BK channels heterologously expressed in Xenopus laevis oocytes.

The electrical current through the BK channel is measured using conventional two-electrode voltage clamp. BK currents are activated by repeating ramp protocols. In brief, the membrane potential is continuously changed from −120 mV to +120 mV within 2 s. The threshold for BK activation is approximately +30 mV under control conditions. Compounds are applies for 100 s during which the ramp protocol is repeated 10 times with 10 s intervals. In between the ramp protocols the membrane potential is clamped at −80 mV. The first three compound applications are control blanks where the current level is allowed to stabilize. In the subsequent 8 applications increasing concentrations (0.01-31.6 μM) of compound is applied and a marked increase in the current level at depolarizing potentials is observed.

In order to evaluate the ability of the compounds to shift the BK activation curve towards lower membrane potentials, the BK current was transformed into conductance by using Ohm's law g=I/(E_{memb}−Eᵣₑᵥ), where g is the conductance, I is the current, E_{memb} is the membrane potential and Eᵣₑᵥ is the reversal potential. The extracellular solution for these experiments contains 2.5 mM K⁺and the intracellular K⁺concentration of an oocyte is estimated to be 100 mM. Under those conditions, Nernst equation predicts a reversal potential of Eᵣₑᵥ=−93.2 mV. The control conductance level at a membrane potential of +100 mV was calculated, and the compound effect was evaluated as the potential difference, ΔV, to the membrane potential at which the same conductance level was obtained in the presence of compound.

The concentration response curve for this potential difference was fitted to the sigmoidal logistic equation: ΔV=ΔVₘₐₓ/(1+(EC₅₀/[compound])ⁿ), where ΔVₘₐₓ represents the maximal left shift of the BK activation curve, EC₅₀ is the concentration causing a half maximal response, and n is the slope coefficient.

The calculated EC50 values for compounds 1, 2 and 9 are 4.4 μM, 2.4 μM and 0.97 μM, respectively. The corresponding ΔVₘₐₓ values for the three compounds are −88 mV, −94 mV and −99 mV, respectively.

1. An aromatic heterocyclic carboxylic acid amide derivative of Formula I 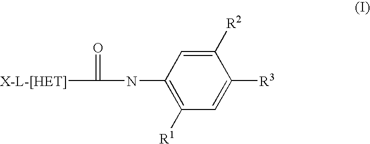
a stereoisomer or a mixture of its stereoisomers, or a pharmaceutically-acceptable addition salt thereof, wherein
X represents alkyl, cycloalkyl, alkenyl, alkynyl, methoxyiminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl, piperidinyl, morpholin-4-yl and pyridinyl, which phenyl and heterocyclic group are optionally substituted one or more times with substituents selected from the group consisting of alkyl, halo, trifluoromethyl, cyano, hydroxy, phenyl and alkoxy-carbonyl;
L is absent (i.e. representing a covalent bond), or represents the linking group —CH₂— or —CONH—;
HET represents a five-membered aromatic heterocyclic group selected from furanyl, thienyl, oxazolyl, thiazolyl, pyrazolyl and triazolyl, which heterocyclic group is optionally substituted one or more times with substituents selected from the group consisting of alkyl, halo, trifluoromethyl and cyano;
R¹ represents tetrazolyl, tetrazolyl-alkoxy, N-phenyl-carbamoyl, alkyl-sulfonyl-amino-carbonyl, N-alkyl-sulfonyl-carboxamide, N-phenyl-sulfonyl-carboxamide, carboxy, N-cyano-carboxamide, sulfamoyl, sulfonic acid, sulfonic acid alkyl ester, sulfonic acid phenyl ester, or a oxadiazolyl oxo- or thio-derivative selected from 5-oxo-4,5-dihydro-[1,2,4]oxadiazol-3-yl and 5-thioxo-4,5-dihydro-[1,2,4]oxadiazol-3-yl;
R² represents halo or trifluoromethyl; and
R³ represents hydrogen, halo, trifluoromethyl, amino, N-alkyl-amino, N,N-dialkyl-amino, piperidinyl or morpholinyl.

2. The aromatic heterocyclic carboxylic acid amide derivative of claim 1, or a pharmaceutically-acceptable addition salt thereof, wherein X represents alkyl, cycloalkyl, alkenyl, alkynyl, methoxy-iminomethyl, amino, N-alkyl-amino, N,N-dialkyl-amino, N-aryl-amino, N,N-diaryl-amino, alkyl-sulfonyl-amino, aryl-sulfonyl-amino, alkyl-carbonyl-amino, aryl-carbonyl-amino, phenyl or a five- or six-membered heterocyclic group selected from imidazolyl, 2-oxopyrrolidin-1-yl, piperidinyl, morpholin-4-yl and pyridinyl, which phenyl and heterocyclic groups are optionally substituted one or more times with substituents selected from the group consisting of alkyl, halo, trifluoromethyl, cyano, hydroxy, phenyl and alkoxy-carbonyl.

3. The aromatic heterocyclic carboxylic acid amide derivative of either one of claims 1-2, or a pharmaceutically-acceptable addition salt thereof, wherein L is absent (i.e. representing a covalent bond), or represents the linking group —CH₂— or —CONH—.

4. The aromatic heterocyclic carboxylic acid amide derivative of any one of claims 1-3, or a pharmaceutically-acceptable addition salt thereof, wherein HET represents a five-membered aromatic heterocyclic group selected from furanyl, thienyl, oxazolyl, thiazolyl, pyrazolyl and triazolyl, which heterocyclic group is optionally substituted one or more times with substituents selected from the group consisting of alkyl, halo and trifluoromethyl.

5. The aromatic heterocyclic carboxylic acid amide derivative of any one of claims 1-4, or a pharmaceutically-acceptable addition salt thereof, wherein R¹ represents tetrazolyl, tetrazolyl-alkoxy, N-phenyl-carbamoyl, alkyl-sulfonyl-amino-carbonyl, N-alkyl-sulfonyl-carboxamide, N-phenyl-sulfonyl-carboxamide, carboxy, N-cyano-carboxamide, sulfamoyl, sulfonic acid, sulfonic acid alkyl ester, sulfonic acid phenyl ester, or a oxadiazolyl oxo- or thio-derivative selected from 5-oxo-4,5-dihydro-[1,2,4]oxadiazol-3-yl and 5-thioxo-4,5-dihydro-[1,2,4]oxadiazol-3-yl.

6. The aromatic heterocyclic carboxylic acid amide derivative of any one of claims 1-5, or a pharmaceutically-acceptable addition salt thereof, wherein R² represents halo or trifluoromethyl.

7. The aromatic heterocyclic carboxylic acid amide derivative of any one of claims 1-6, or a pharmaceutically-acceptable addition salt thereof, wherein R³ represents hydrogen, halo, trifluoromethyl, amino, N-alkyl-amino, N,N-dialkyl-amino, piperidinyl or morpholinyl.

8. The aromatic heterocyclic carboxylic acid amide derivative of claim 1, which is
5-(4-Chloro-phenyl)-2-trifluoromethyl-furan-3-carboxylic acid [5-chloro-2-(1H-tetrazol-5-yl)-phenyl]-amide;
1-(4-Chloro-phenyl)-5-trifluoromethyl-1H-pyrazole-4-carboxylic acid [5-chloro-2-(1H-tetrazol-5-yl)-phenyl]-amide;
1-(4-Bromo-benzyl)-5-methyl-1H-[1,2,3]triazole-4-carboxylic acid [5-chloro-2-(1H-tetrazol-5-yl)-phenyl]-amide;
5-(4-Chloro-phenyl)-2-trifluoromethyl-furan-3-carboxylic acid (5-chloro-2-phenylcarbamoyl-phenyl)-amide;
5-(4-Chloro-phenyl)-2-trifluoromethyl-furan-3-carboxylic acid (5-chloro-2-methanesulfonylaminocarbonyl-phenyl)-amide, 4-Chloro-2-{[5-(4-chloro-phenyl)-2-trifluoromethyl-furan-3-carbonyl]-amino}-benzenesulfonic acid;
5-(4-Chloro-phenyl)-2-trifluoromethyl-furan-3-carboxylic acid [5-chloro-2-(1H-tetrazol-5-ylmethoxy)-phenyl]-amide;
4,5-Dichloro-2-{[5-(4-chloro-phenyl)-2-trifluoromethyl-furan-3-carbonyl]-amino}-benzenesulfonic acid;
4-Methyl-2-(4-trifluoromethyl-phenyl)-thiazole-5-carboxylic acid [5-chloro-2-(1H-tetrazol-5-yl)-phenyl]-amide;
4-Chloro-2-{[5-(4-chloro-phenyl)-2-trifluoromethyl-furan-3-carbonyl]-amino}-benzoic acid;
4-Chloro-2-{[5-(4-chloro-phenyl)-2-trifluoromethyl-furan-3-carbonyl]-amino}-benzoylcyanamide;
5-(4-Chloro-phenyl)-2-methyl-furan-3-carboxylic acid [5-chloro-2-(1H-tetrazol-5-yl)-phenyl]-amide;
2-{[5-(4-Chloro-phenyl)-2-trifluoromethyl-furan-3-carbonyl]-amino}-4,5-difluoro-benzoic acid;
4,5-Dichloro-2-{[5-(4-chloro-phenyl)-2-trifluoromethyl-furan-3-carbonyl]-amino}-benzenesulfonic acid methyl ester;
5-Methyl-2-phenyl-2H-[1,2,3]triazole-4-carboxylic acid [5-chloro-2-(1H-tetrazol-5-yl)-phenyl]-amide;
5-Trifluoromethyl-2-(4-trifluoromethyl-phenyl)-oxazole-4-carboxylic acid [5-chloro-2-(5-oxo-4,5-dihydro-[1,2,4]oxadiazol-3-yl)-phenyl]-amide;
5-Trifluoromethyl-2-(4-trifluoromethyl-phenyl)-oxazole-4-carboxylic acid [5-chloro-2-(1H-tetrazol-5-yl)-phenyl]amide;
5-(4-Chloro-phenyl)-2-trifluoromethyl-furan-3-carboxylic acid [5-chloro-2-(5-oxo-4,5-dihydro-[1,2,4]oxadiazol-3-yl)-phenyl]-amide;
5-(4-Chloro-phenyl)-2-trifluoromethyl-furan-3-carboxylic acid [4,5-dichloro-2-(1H-tetrazol-5-yl)-phenyl]-amide;
5-(4-Chloro-phenyl)-2-methyl-furan-3-carboxylic acid [5-chloro-2-(5-oxo-4,5-dihydro-[1,2,4]oxadiazol-3-yl)-phenyl]-amide;
2-(4-Chloro-benzyl)-thiazole-4-carboxylic acid [5-chloro-2-(5-oxo-2,5-dihydro-[1,2,4]oxadiazol-3-yl)-phenyl]-amide;
1-(4-Bromo-benzyl)-5-methyl-1H-[1,2,3]triazole-4-carboxylic acid [5-chloro-2-(5-oxo-4,5-dihydro-[1,2,4]oxadiazol-3-yl)-phenyl]-amide;
4-Methyl-2-morpholin-4-yl-thiazole-5-carboxylic acid [5-chloro-2-(5-oxo-4,5-dihydro-[1,2,4]oxadiazol-3-yl)-phenyl]-amide;
4-Methyl-2-morpholin-4-yl-thiazole-5-carboxylic acid [5-chloro-2-(1H-tetrazol-5-yl)-phenyl]-amide;
2-(3-Chloro-phenyl)-5-methyl-2H-[1,2,3]triazole-4-carboxylic acid [5-chloro-2-(5-oxo-4,5-dihydro-[1,2,4]oxadiazol-3-yl)-phenyl]-amide;
4-{5-[5-Chloro-2-(5-oxo-4,5-dihydro-[1,2,4]oxadiazol-3-yl)-phenylcarbamoyl]-4-methyl-thiazol-2-yl}-piperidine-1-carboxylic acid tert-butyl ester;
4-Methyl-2-(4-trifluoromethyl-phenyl)-thiazole-5-carboxylic acid [5-chloro-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-4-morpholin-4-yl-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-4-dimethylamino-2-(5-oxo-4,5-dihydro-[1,2,4]oxadiazol-3-yl)-phenyl]-amide;
5-(4-Chloro-phenyl)-2-trifluoromethyl-furan-3-carboxylic acid [5-chloro-2-(5-oxo-4,5-dihydro-[1,2,4]oxadiazol-3-yl)-4-piperidin-1-yl-phenyl]-amide;
2-(3-Chloro-phenyl)-5-methyl-2H-[1,2,3]triazole-4-carboxylic acid [5-chloro-2-(1H-tetrazol-5-yl)-phenyl]-amide;
4-Methyl-2-pyridin-4-yl-thiazole-5-carboxylic acid [5-chloro-2-(1H-tetrazol-5-yl)-phenyl]-amide;
4-Methyl-2-(4-trifluoromethyl-phenyl)-thiazole-5-carboxylic acid [5-chloro-2-(5-oxo-4,5-dihydro-[1,2,4]oxadiazol-3-yl)-4-piperidin-1-yl-phenyl]-amide;
5-(Cyclopentanecarbonyl-amino)-3-methyl-thiophene-2-carboxylic acid [5-chloro-2-(1H-tetrazol-5-yl)-phenyl]amide; or
2,4-Dichloro-6-[(4-methyl-2-morpholin-4-yl-thiazole-5-carbonyl)-amino]-benzoic acid;
or a pharmaceutically-acceptable addition salt thereof.

9. A pharmaceutical composition comprising a therapeutically effective amount of the aromatic heterocyclic carboxylic acid amide derivative of any one of claims 1-8, or a pharmaceutically-acceptable addition salt thereof, or a prodrug thereof, together with one or more adjuvants, excipients, carriers and/or diluents.

10. Use of an aromatic heterocyclic carboxylic acid amide derivative of any one of claims 1-8, or a pharmaceutically-acceptable addition salt thereof, for the manufacture of a pharmaceutical composition/medicament for the treatment, prevention or alleviation of a disease or a disorder or a condition of a mammal, including a human, which disease, disorder or condition is responsive to modulation of potassium channels.

11. The use according to claim 10, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhea, ocular hypertension (glaucoma) or baldness.

12. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the aromatic heterocyclic carboxylic acid amide derivative according to any one of claims 1-8.
